(19) United States
(12) Patent Application Publication
(10) Pub. No.: US 2012/0108538 A1
(43) Pub. Date: May 3, 2012

(54) SULFATED POLYSACCHARIDES HAVING ANTIPLASMODIAL ACTIVITY AND METHODS AND PRODUCTS FOR IDENTIFYING ANTIPLASMODIAL ACTIVITY

(76) Inventors: James Beeson, Victoria (AU); Michelle Boyle, Victoria (AU); Jonathan Richards, Victoria (AU); Danny Wilson, Victoria (AU)

(21) Appl. No.: 13/381,684

(22) PCT Filed: Jun. 29, 2010
(86) PCT No.: PCT/AU2010/000813

§ 371 Date: Dec. 30, 2011

Related U.S. Application Data
(60) Provisional application No. 61/221,620, filed on Jun. 30, 2009.

(51) Int. Cl. A61K 31/737; A61P 33/06; C12Q 1/18; C08B 37/00

(52) U.S. Cl. 514/54; 536/118; 435/32

(57) ABSTRACT

This invention relates to methods for identifying, producing or rationally designing sulfated polysaccharide molecules that have antiplasmodial activity. Also provided are sulfated polysaccharide molecules having antiplasmodial activity, as well as methods for treating and preventing diseases including malaria with such molecules.

### FIELD OF THE INVENTION

The present invention relates to the design, screening, production and use of sulfated polysaccharide molecules for the treatment of Plasmodium spp infection, and particularly Plasmodium falciparum. The invention also relates to methods of identifying antiplasmodial activity and providing products to assist in the identification of the activity.

### BACKGROUND TO THE INVENTION

Human malaria is caused by infection with protozoan parasites of the genus Plasmodium. Five species are known to cause human disease: Plasmodium falciparum, Plasmodium malariae, Plasmodium ovale, Plasmodium vivax and Plasmodium knowlesi. However, Plasmodium falciparum is responsible for the majority of severe disease and death. Recent estimates of the annual number of clinical malaria cases worldwide range from 214 to 397 [1,2], although a higher estimate of 515 million (range 300 to 660 million) clinical cases of Plasmodium falciparum in 2002 has been proposed [3]. Annual mortality (nearly all from Plasmodium falciparum malaria) is thought to be around 1.1 million. Malaria also significantly increases the risk of childhood death from other causes [3]. Almost half of the world's population lives in areas where they are exposed to risk of malaria [4], and the increasing numbers of visitors to endemic areas are also at risk. Despite continued efforts to control malaria, it remains a major health problem in many regions of the world, and new ways to prevent and/or treat the disease are urgently needed. 1. Breman J G, Alilio M S, Mills A (2004) Conquering the intolerable burden of malaria: what's new, what's needed: a summary. Am J Trop Med Hy71: 1-15.2. WHO (2005) World Malaria Report 2005. Geneva: World Health Organisation.3. Snow R W, Korenromp E L, Gouws E (2004) Pediatric mortality in Africa: plasmodium falciparum malaria as a cause or risk? Am J Trop Med Hyg 71: 16-24.4. Hay S I, Guerra C A, Tatem A J, Noor A M, Snow R W (2004) The global distribution and population at risk of malaria: past, present, and future. Lancet Infect Dis 4: 327-336.

While significant attention is directed toward the design of a vaccine against malaria, research is continuing into the identification of therapeutic agents capable of treating an infected subject. For example, it is known in the art that heparin is useful for inhibiting the growth of malaria parasites in vitro. In humans, heparin has historically been used as an experimental treatment of disseminated intravascular coagulation in malaria patients (for example [5,6]) however its use is no longer recommended due to serious bleeding-related side-effects [7]. 5. Mitchell A D (1974) Recent experiences with severe and cerebral malaria. S Afr Med J 48: 1353-1354.6. Munir M, Tjandra H, Rampengan T H, Mustadjab I, Wulur F H (1980) Heparin in the treatment of cerebral malaria. Paediatr Indones 20: 47-50.7. WHO (2006) WHO. Guidelines for the treatment of Malaria. Geneva: World Health Organization. 266 p.

Efforts have been made to alter the structure of heparin to understand structure/function relationships of activity using techniques attempting to selectively de-sulfate specific residues in heparin [8,9]. However, these techniques are apt to de-sulfated at off-target positions, and also to have difficulties of distinguishing the consequences from loss of overall negative charge. These studies have therefore failed to provide definitive information as to the various structure/function relationships in heparin and related molecules. 8. Clark D L, Su S, Davidson E A (1997) Saccharide anions as inhibitors of the malaria parasite. Glycoconj J 14: 473-479.9. Kulane A; Ekre H P, Perlmann P, Rombo L, Wahlgren M, et al. (1992) Effect of different fractions of heparin on Plasmodium falciparum merozoite invasion of red blood cells in vitro. Am J Trop Med Hyg 46: 589-594.

It is an aspect of the present invention to overcome or alleviate a problem of the prior art bjr providing structure/function relationships between sulfated polysaccharides to provide new and advantageous agents for the treatment of malaria; The relationships further provide tools for the design of antiplasmodial agents which target malaria heparin binding proteins of Plasmodium that are involved in growth of the parasite.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, the present invention provides a method for identifying antiplasmodial activity in a candidate sulfated polysaccharide molecule, the polysaccharide molecule comprising two or more disaccharide units, the method comprising one or more of the following steps: (i) assessing the average degree of sulfation per disaccharide unit, (ii) assessing the position of two or more sulfate groups in at least one of the disaccharide units, (iii) assessing the linkage of one or more sulfate groups in at least one of the disaccharide units, (iv) assessing the saccharide backbone composition, wherein the candidate molecule is considered to possess antiplasmodial activity if one or more of the following conditions is satisfied: (a) the average degree of sulfation is at least about 1 sulfate group per disaccharide unit (b) 2 or more sulfate groups are present on a single monosaccharide residue of the disaccharide unit (c) 50% or more of sulfate groups are O-linked, (d) the saccharide backbone composition comprises 50% or less of iduronic acid.

Thus, the candidate molecule is considered to possess antiplasmodial activity if one or more of the following conditions is satisfied: (a) the average degree of sulfation is at least about 1 sulfate group per disaccharide unit (b) 2 or more sulfate groups are present on a single monosaccharide residue of the disaccharide unit (c) 50% or more of sulfate groups are O-linked (d) the saccharide backbone composition comprises 50% or less of iduronic acid. The method of identifying may comprise any two or more of steps (i), (ii), (iii) or (iv). In another embodiment, the method comprises any three or more of steps (i), (ii), (iii) or (iv). In a further embodiment the method comprises steps (i), (ii), (iii) and (iv).

In a second aspect the present invention provides a method for producing or rationally designing a sulfated polysaccharide having antiplasmodial activity, the method comprising the steps of providing a polysaccharide molecule or a sulfated polysaccharide molecule having two or more disaccharide units, modifying the polysaccharide or sulfated polysaccharide molecule by one or more of the following methods: (i) alter or ensure the average degree of sulfation is at least about 1 sulfate group per disaccharide unit, (ii) alter or ensure the position of sulfation is such that 2 or more sulfate groups are present on a single monosaccharide residue of a disaccharide unit, (iii) alter or ensure the linkage of sulfation is such that 50% or more of sulfate groups are O-linked (iv) alter or ensure that the disaccharide backbone composition comprises 50% or less iduronic acid. The method of producing or rationally designing the sulfated polysaccharide may comprise any two or more of steps (i), (ii), (iii) or (iv). In another embodiment, the method comprises any three or more of steps (i), (ii), (iii) or (iv). In a further embodiment the method comprises steps (i), (ii), (iii) and (iv).

In the methods of identifying, producing or rationally designing a sulfated polysaccharide molecule, the average degree of sulfation may be at least about 1, 1.5, 2, 2.5, 3, 3.5, 4, 4.5 or 5 sulfate groups per disaccharide unit. The polysaccharide molecule may be considered to have antiplasmodial activity if 50% or more of sulfate groups are O-linked, or the identity of at least one residue of the polysaccharide molecule is uronic acid or hexosamine. Preferably, the sulfated polysaccharide molecule may be considered to have antiplasmodial activity if, additionally, the saccharide backbone comprises 50% or less of iduronic acid.

The sulfated polysaccharide may consist of at least 2-10,000 disaccharide units. Thus in one aspect, the sulfated polysaccharide may consist of 2-10, 10-20, 20-40, 40-80, 80-150, 150-500, 500-1000, 1000-5000 or 5000-10000 disaccharide units. Preferably, the sulfated polysaccharide may consist of at least 2, 3, 4, 5, 6, 7, 8, 9 or 10 disaccharide units. Thus, the sulfated polysaccharide may have a molecular weight of up to 1000 kDa.

In a further aspect the present invention provides a sulfated polysaccharide molecule identified, produced or rationally designed by a method described herein.

A further aspect of the invention provides a composition comprising a sulfated polysaccharide molecule identified, produced or rationally designed, as described herein and a pharmaceutically acceptable carrier.

A further aspect of the present invention provides a method for treating or preventing an infection with a Plasmodium, the method comprising administering to a subject in need thereof an effective amount of a composition as described herein, wherein the sulfated polysaccharide molecule is not a compound selected from the group consisting of heparin, heparin sulfate, pentosan polysulfate, dextran sulfate, curdlan sulfate, cellulose sulfate, a carrageen, periodate treated heparin, and fucoidan. The Plasmodium may be Plasmodium falciparum, Plasmodium malariae, Plasmodium ovale, Plasmodium vivax and Plasmodium knowlesi. Typically, the Plasmodium is Plasmodium falciparum.

A further aspect of the present invention provides a method of at least partially synchronising a population of two or more Plasmodium-infected cells comprising exposing the two or more Plasmodium-infected cells to a protease inhibitor to inhibit schizont rupture, thereby halting development of the Plasmodium-infected cells at the schizont stage.

In a further aspect, the present invention provides a method of isolating Plasmodium merozoites from an at least partially synchronous population produced by the method as described herein comprising rupturing the schizonts to allow merozoite release.

In yet another aspect, the present invention provides a population of substantially synchronised Plasmodium-infected cells.

In another aspect, the invention provides a population of merozoites from a population of Plasmodium-infected cells wherein the cells are desirably substantially synchronised and the population of merozoites has 50% 60%, 65%, 70%, 75%, 80%, 85%, 90%, 91%, 92%, 93%, 94%, 95%, 96%, 97%, 98%, 99%, 99.5% or more purity.

In yet a further aspect, the present invention provides a method for identifying a compound capable of inhibiting invasion of one or more Plasmodium merozoites into one or more cells, the method comprising exposing the one or more cells to the one or more merozoites in the presence of the candidate compound and determining whether invasion of the one or more merozoites into the one or more cells has occurred. In one embodiment, the candidate compound is a sulfated polysaccharide molecule.

### BRIEF DESCRIPTION OF THE DRAWINGS

Some figures contain coloured representations or entities. Coloured versions of the figures are available from the Patentee upon request or from an appropriate Patent Office. A fee may be imposed if obtained from a Patent Office.

TABLE 1 shows structure and sulfation patterns of exemplary polymeric carbohydrates compounds. The panel of compounds tested for inhibitory activity include (A) modified heparin compounds, (B) E. Coli K5 capsular polysaccharide derivatives, (C) and chondroitin sulfate (CS) molecules. Compounds have a backbone composition of iduronic (IdoA (orange underline) or glucuronic acid. (GlcA) and glucosamine (GlcN) or galactosamine (GalN) in disaccharide units. Compounds are sulfated at various points including O-sulfation of uronic acid at carbon 2 (2S, yellow) or carbon 3 (3S, blue) and of amino groups at carbon 3 (3S, purple), carbon 4 (4S, orange) and carbon 6 (6S, red) and sulfation of the nitrogen of the amino group (NS, green). For sulfation levels of exemplary compounds, refer to Table 2.

TABLE 2 shows the inhibitory activity of different heparin-like compounds against P. falciparum blood stage growth. IC₅₀ values are calculated via linear regression from dose-dependent inhibitory curves calculated in growth inhibitory assays. D.o.s is degree of sulfation from manufacturer's descriptions of compounds referring to the average number of sulfate groups per di-saccharide unit of carbohydrate. Sulfation levels were obtained from the manufacturer's and published data [10-13]. In table A, column 3 shows the proportion of disaccharide units that have sulfation of the uronic acid residue; column 4 shows the proportion of disaccharide units that have sulfation of the hexosamine residue (the specific position of the sulfate groups is also indicated). In Table B, sulfate level is firstly described as % of disaccharide unit with singular sulfation, and then % of di-saccharide units with di-sulfation across di-saccharide. All compounds in Table A contain Glucosamine (GlcNAc) as the Hexosamine group. All compounds in Table B contain Galactosamine (GalNAc) as the Hexosamine group. * K5-OS(H) also contains 3-O-sulfation of the amino residue of unknown level (Table 1A). Abbreviations: IdoA: iduronic acid, GlcA: glucuronic acid, K5-NS: K5 polysaccharide N sulfated, K5-OS-L: K5 polysaccharide low level O sulfated, K5-OS-H: K5 polysaccharide high level O sulfated, K5-NSOS-L: K5 polysaccharide low level N and O sulfated, K5-NSOS-H: K5 polysaccharide high level N and O sulfated, EK5-NSOS-L: epimerized K5 polysaccharide low level N and O sulfated, EK5-NSOS-H: epimerized K5 polysaccharide high level N and O sulfated, CSC: chondroitin sulfate C, CSD: chondroitin sulfate D, CSE: chondroitin sulfate E, chondroitin sulfate B-2-6-di-sulfated, CSB-2,4-OS, chondroitin sulfate B-2,4-di-sulfated. 10. Alkhalil A, Achur R N, Valiyaveettil M, Ockenhouse C F, Gowda D C (2000) Structural requirements for the adherence of Plasmodium falciparum-infected erythrocytes to chondroitin sulfate proteoglycans of human placenta. J Biol Chem 275: 40357-40364.11. Bergefall K, Trybala E; Johansson M, Uyama T, Naito S, et al. (2005) Chondroitin sulfate characterized by the E-disaccharide unit is a potent inhibitor of herpes simplex virus infectivity and provides the virus binding sites on gro2C cells. J Biol Chem 280: 32193-32199.12. Kinoshita A, Yamada S, Haslam S M, Morris H R, Dell A, et al. (1997) Novel tetrasaccharides isolated from squid cartilage chondroitin sulfate E contain unusual sulfated disaccharide units GlcA(3-O-sulfate)beta1-3GalNAc(6-O-sulfate) or GlcA(3-O-sulfate)beta1-3GalNAc. J Biol Chem 272: 19656-19665.13. Leali D, Belleri M, Urbinati C, Coltrini D, Oreste P, et al. (2001) Fibroblast growth factor-2 antagonist activity and angiostatic capacity of sulfated Escherichia coli K5 polysaccharide derivatives. J Biol Chem 276: 37900-37908.

TABLE 3 shows antibodies and compounds tested for inhibition of invasion or schizont rupture.

FIG. 1 shows the specificity and reproducibility of inhibitory activity of heparin against P. falciparium blood stage growth. (A) Heparin and CSC were tested for inhibition of P. falciparum blood stage growth in one (48 hrs) and two (72 hrs) cycle growth inhibition assays (GIAs). (B) Reproducibility of GIAs was analysed by comparing heparin inhibition over one cycle of P. falciparum blood stage growth in three repeated experiments. Data are mean growth±s.e.m., expressed as percent of control (HT-PBS) (two assays in duplicate for two cycle assays; >4 times assays in duplicate for one cycle assays). 3D7 parasite line was used in all experiments. (Abbreviations: CSC—chondroitin sulfate C, GAG—glycosaminoglycan, GIA—growth inhibitory assay.)

FIG. 2 shows the inhibition of P. falciparum blood stage growth by heparin and related compounds. (A) Heparin and related compounds dextran sulfate, fucoidan, K5-NSOS-H and CSC were tested for inhibition of P. falciparum blood stage growth in GIAs. (B) Heparin and fondaparinux (obtained from GlaxoSmithKline), a synthetic pentasaccharide synthetic compound based on the antithrombin binding motif of heparin were tested for inhibitory activity. Non-inhibitory CSC was included in assays as a negative control, (data removed for clarity). Data are mean growth ±s.e.m., expressed as percent of control (HT-PBS) (three assays in duplicate). 3D7 parasite line was used in all experiments. (Abbreviations: CSC—chondroitin sulfate C, GIA—growth inhibitory assay, K5-NSOS-H—K5 high level N and O-sulfated.)

FIG. 3 shows the importance of sulfate groups for the inhibitory activity of heparin. Modified heparin compounds, de-N-sulfated and de-N/O-sulfated heparin, were tested alongside heparin for inhibition of P. falciparum blood stage growth in GIAs. Non-inhibitory CSC was included in assays as a negative control, (data removed for clarity). Data are mean growth±s.e.m., expressed as percent of control (HT-PBS) (two assays in duplicate). 3D7 parasite line was used in all experiments. (Abbreviations: CSC—chondroitin sulfate, GIA—growth inhibitory assay.)

FIG. 4 shows that level and pattern of sulfation, and the chain length, of heparin like compounds is involved in activity. A shows the importance of level and pattern of N and O sulfation for inhibitory activity of heparin. Fully de-N-sulfated heparin and partially de-N-sulfated heparin, de-N/O-sulfated heparin, and de-6-O-sulfated and de-2-O-sulfated heparin were tested alongside unmodified heparin for inhibition of P. falciparum blood stage growth in GIAs. B) shows that N sulfation is not an absolute requirement for activity—N or O sulfated K5 polysaccharides, K5-NS, K5-OS-L and K5-OS-H were tested for inhibition of P. falciparum blood stage growth in GIAs. C) shows that pattern and level of sulfation affect inhibitory activity but that IdoA is not required—N and O sulfated K5 polysaccharides K5-NSOS-L, K5-NSOS-H and their epimerised derivatives EK5-NSOS-L and EK5-NSOS-H were tested for inhibition in GIAs. D) shows size-dependent inhibition of P. falciparum blood stage growth by heparin oligosaccharides. Heparin and CSC oligosaccharides at 100 μg/mL were tested in GIAs for inhibitory activity against the 3D7 parasite line. For all figures CSC non-inhibitory compound was included in assays as a negative control, (data removed for clarity). Data are mean growth±s.e.m., expressed as percent of control (HT-PBS) (three assays in duplicate, except for D, two experiments in duplicate). 3D7 parasite line was used in all experiments.

FIG. 5. shows that specific chondroitin sulfates have inhibitory activity against P. falciparum in GIAs, confirming that inhibitory activity of sulfated polysaccharides is dependent of sulfation levels and patterns and not the form of the hexosamine. Chondroitin sulfate compounds, CSD, CSE and CSB-2,4-OS and CSB-2,6-OS were tested for inhibitory activity in GIAs against P. falciparum.

FIG. 6. shows inhibition of different P. falciparum lines. (A) Heparin, (B) CSE, (C) K5-NS,OS-H, and (D) CSC were tested for inhibition of P. falciparum blood stage growth using three different parasite lines, 3D7, D10, W2mef in GIAs. Data are mean growth±s.e.m., expressed as percent of control (HT-PBS) (three assays in duplicate). (Abbreviations: CSC—chondroitin sulfate C, CSE chondroitin sulfate E, GIA—growth inhibitory assay, K5-NS,OS-H—K5 high level N and O sulfated). Statistical comparison, 3D7/D10 heparin 50 μg/mL; P=0.007, Mann-Whitney U sample test.

FIG. 7 shows that heparin causes slight delay of schizont rupture and effectively inhibits merozoite invasion. The time course of schizont rupture (A) and ring formation (B). Trophozoite stage cultures of ˜4% parasitemia were incubated with heparin 100 μg/mL or PBS control. At late schizont stage (˜48 hrs of lifecycle), parasites were stained with ethidium bromide and analyzed using flow cytometry. Time points were taken every 3 hrs for 15 hrs and analyzed for late trophozoite/schizont stages and ring stage infected erythrocytes. Data are mean±range of one representative assay (two assays were performed in duplicate). * P=0.03 Difference in schizont parasitemia for heparin versus control at 15 hrs (Figure. A; Mann-Whitney U test). C, D. Live video microscopy was used to observed merozoite invasion events in real time. C. Merozoite invasion in control cultures (see arrow): (1) initial contact of merozoite with erythrocyte, (2) reorientation (3) commencement of invasion, (3-5) mechanical invasion and deformation of the erythrocyte, (6) complete invasion and erythrocyte reformation. D. Merozoite invasion with cultures containing heparin 100 μg/mL: (1-5) merozoite initial contact with erythrocyte with no reorientation, (6) initial contact is not sustained and merozoite detach. No invasion, reorientation or erythrocyte deformation observed in the presence of heparin. Time in seconds is indicated in bottom right corner. Pictures are selected from live video microscopy recordings using the 3D7 P. falciparum line.

FIG. 8 shows that heparin inhibits multiple invasion pathways and does not induce resistance in parasites over one or two selection cycles. Heparin was tested for inhibition of P. falciparum using different invasion pathways A: Heparin effectively inhibited P. falciparum lines W2mef (sialic-acid dependent invasion phenotype) and 3D7 (sialic-acid independent invasion phenotypes). B. Heparin had similar inhibitory activity against invasion of 3D7 P. falciparum into neuraminidase, chymotrypsin, or trypsin-treated erythrocytes versus untreated erythrocytes. C. Ability of heparin to select for resistant parasites was investigated; 3D7 parasites were selected with heparin at 100 μg/mL over 48 hrs. Surviving parasites were cultured as normal until reaching high parasitemia (>5%) and the selection repeated. Parent, heparin selected once (heparin selected—1) and heparin selected twice (heparin selected—2) parasite cultures were tested in GIAs for inhibition by heparin. There was no significant difference in the inhibition of parasites with and without selection. All data are mean growth±s.e.m., expressed as percent of control (HT-PBS) (two or three assays in duplicate).

FIG. 9 shows heparin IC₅₀ is reduced when used in combination with other inhibitors, pyrimethamine (A) and AMA1 binding peptide (B). Heparin was tested in combination with pyrimethanine and AMA1-binding peptide for inhibition of P. falciparum blood stage growth in GlAs. A. Increasing concentrations of pyrimethamine (nM) were tested together with heparin. B. Increasing concentration of AMA1 binding peptide (pg/mL) were tested together with heparin. Data are mean growth±s.e.m., expressed as percent of control

FIG. 10 shows structure of heparin and related compounds. A. Heparin—the major disaccharide unit of heparin is of 4-linked beta-glucuronic acid (GlcA) and alpha-N-acetyl glucosamine (GlcNAc). The major disaccharide is epimerized, 2-O-sulfated at the IdoA and N and 6-O-sulfated at the GlcNAc. Heparin can be modified to be selectively de-sulfated from N (re-acetyl) and/or O positions; B. K5 polysaccharide—has the same backbone as the precursor to heparin and can be modified with the addition of sulphate groups at indicated positions (arrows) and with the epimerization of HexA from GlcA to IdoA (indicated in grey) resulting in polysaccharides with various sulfation and backbone compositions. C. Chondroitin sulfate (CS)—backbone composition of GlcA or IdoA (grey) with HexA-[1-3]-GalNAc-[1-4]. Possible O-linked sulfate groups are indicated with arrows.

FIG. 11 shows native MSP1-42 but not full length heparin or EBA175 binds to heparin. Merozoite proteins extracted from whole schizonts in TX100 were bound to heparin immobilized on agarose with and without soluble inhibitors. Bound proteins were eluted with reducing sample buffer and analysed via SDS-PAGE and western blotting with anti-MSP1 antibodies (MSP1-19 fragment) and anti-EBA175 antibodies. A. MSP1-42 (see arrow) was able to bind to immobilized heparin in the presence of HT-PBS (control) and soluble CSC but not soluble heparin, being depleted from the unbound supernatant fraction. Full length MSP1 (MSP1-f, arrow) was not able to bind, only present in unbound supernatant. B. EBA175 was not able to bind heparin-agarose, with the majority only seen the unbound supernatant fractions in control and soluble heparin inhibited binding assays. C. Binding of MSP1-42 to heparin-agarose was specific, with de-N-sulfated and de-60-sulfated unable to inhibit binding. D. Specificity of MSP1-42 binding to heparin correlated with GIA activity of K5-polysacharides, with only K5-NSOS-H being able to clearly inhibit binding. MSP1-42, MSP1-full length and EBA175 are indicated with arrows. Ladder markers as indicated. Data is representative of repeated assays.

FIG. 12 shows recombinant MSP1-42, but not MSP1-19 or AMA1 bind to heparin. A: Recombinant merozoite antigens were tested in heparin-BSA binding assays for binding, to heparin-BSA (5 μg/mL) conjugate and BSA (5 μg/mL) alone. Binding to lactoferrin was used as a positive control and to normalize, binding between assays. Data are mean binding±s.e.m. expressed as percent binding of lactoferrin (three assays in duplicate). Heparin-BSA had similar inhibitory activity against P. falciparum in vitro growth in GlAs as heparin, while BSA is not inhibitory (data not shown). B. Binding of heparin to MSP1-42 is dose dependent and saturable. Data are normalized to percent binding of lactoferrin to heparin-BSA at 5 μg/mL. C. Binding of heparin-BSA to MSP1-42 was inhibited with soluble heparin but not CSC. Data are normalized to binding of uninhibited. Data are mean±s.e.m., expressed as percent binding of uninhibited controls (HT-PBS) (three assays in duplicate).

FIG. 13 shows models of the structural requirements of heparin for inhibitory activity. A. Correlation between IC50 and d.o.s. of inhibitory compounds shows that a d.o.s. of 1.5 or higher is required for activity. Non-inhibitory compounds de-N/O-sulfated heparin, de-N-sulfated heparin, K5-NS, and K5-OS-L are plotted here as IC₅₀=500 for clarity, however none of these compounds showed any inhibition at this concentration. B. Carbohydrate modelling program Sweet II was used to generate ball-and-stick and relative molecular lipophilicity potential models of inhibitory and non-inhibitory compounds. Models were based on a hexasaccharide sequence.

FIG. 14 shows purification of merozoites and invasion of RBCs. A. Representative flow cytometry plot showing different cell populations of free merozoites, uninfected RBCs, RBCs with bound merozoites and infected RBCs. Note, this plot shows no infected RBCs (see figure C right panel and FIG. 17 for FACS plots containing infected RBCs). B, C: Filtration effectively purifies viable merozoites from E64-treated schizonts. FACS plots (B) and Giemsa-stained smears (C) show merozoites of high purity after filtration of E64-treated schizonts (left panels), and that purified merozoites were able to bind and invade RBCs (middle panel), resulting in a highly synchronous population of intra-erythrocytic parasites (right panel). D. Surface labelling of purified merozoites with antibodies to AMA1 (red; counter stained with DAPI (blue) to label the merozoites nucleus). E. Labelling of the rhoptry with antibodies to RAP1. F. Transmission electron micrograph of purified merozoites labelled to identify key structures.

FIG. 15 shows kinetics and requirements for merozoites invasion. A. The proportion of merozoites that invade RBCs (invasion rate) is affected by the ratio of merozoites to RBCs. As the ratio of merozoites to RBCs increases, the invasion rate of merozoites decreases. Data is representative of four assays in duplicate. B. The invasive potential of merozoites declines over time, and is affected by different temperatures. Merozoites were incubated at 37° C., 22° C., or on ice after purification before being mixed with RBCs to measure invasion. Data is mean +/− SEM of 7 assays in duplicate. C. The rate of merozoite invasion over time is rapid following incubation with uninfected RBCs. The proportion of merozoites that have invaded with increasing time is shown as a percent of maximum invasion recorded in non-inhibited samples. Data is mean +/− range of two assays in duplicate. D. Merozoite invasion occurs in the presence and absence of serum components. Merozoites were tested for the ability to invade RBCs in the presence of serum at various concentrations. Serum was used with and without dialysis against RPMI-HEPES. Data is mean +/− SEM of three assays in duplicate and expressed as a percent of invasion into RMPI-HEPES alone.

FIG. 16 shows the development of an invasion inhibition assay using purified merozoites. A. Various inhibitory and non-inhibitory compounds and antibodies were tested for their ability to inhibit invasion of purified merozoites in invasion inhibitory assays (IIA). Invasion is expressed as a proportion of control. The concentration of inhibitors is in mg/ml unless otherwise indicated. Data is mean +/− range of two assays in duplicate. B. Compounds were tested for inhibition of schizont rupture by incubating with late stage parasites and measuring parasitemia and schizont rupture by flow cytometry over time. Rupture is expressed as a proportion of control. Data is mean +/− range of two assays in duplicate. C, D, E. Comparison of inhibitory activities in IIA versus conventional growth inhibition assays (GIA) of AMA1-binding peptide R1 (C) and the anti-AMA1 MAbs 1F9 and 2C5 (D) and the invasion inhibitory heparin (E).

FIG. 17 shows the isolation of merozoites and invasion of RBCs. Purification of merozoites and invasion of RBCs was monitored by Geimsa-stained smears (upper panels) and flow cytometry (lower panels). For identification of parasites by flow cytometry, parasites were stained with ethidium bromide (EtBr) and different cell sub-sets were identified by analysis of channels FL2 and FSC. Identification of cell subsets is shown in FIG. 18 A. E64-treatment effectively blocked schizont rupture leading to a parasite population enriched for schizonts. B. Filtration of E64-treated schizonts resulted in a preparation of free merozoites and hemozoin crystals, with no schizonts remaining. C. Purified merozoites mixed with uninfected RBCs. A proportion of merozoites invaded RBCs and others remain bound to the RBC surface. D, E. Cultures resulting from the mixing of purified merozoites and RBCs were followed for 24 hours (D) or 42 hours (E) showing that purified merozoites that had invaded developed normally and led to a highly synchronous parasite culture.

FIG. 18 shows the identification of different RBC and parasite populations by flow cytometry. A. A representative cell plot from flow cytometry analysis to demonstrate the identification of different parasite and RBC populations based on EtBr fluorescence detected in FL2 (which identifies parasites) and cell size (FSC). Note that in this plot no infected RBCs are present; refer to FIG. 17 D and E. B. Image of a GFP-fluorescent merozoite bound to the surface of a RBC that was obtained by cell sorting the population of uninfected RBCs with bound merozoites as shown in (A).

FIG. 19 shows immunofluorescence microscopy of purified merozoites stained with antibodies to different merozoite antigens. Merozoites were stained with antibodies to AMA1 (A), MSP2 (B), MSP1 block 2 (C), and RAP1 (D). Antigen staining is shown with Alexa594 (red), nucleus is stained with DAPI (blue).

FIG. 20 shows merozoites and hemozoin are partially separable by passing over magnet purification columns. Purified merozoites from filtration of E64-treated schizonts were passed over MACs magnet purification columns. A. Preparation of purified merozoites immediately after filtration of E64-treated schizonts. A Geimsa-stained smear of this preparation is also shown, with merozoites and hemozoin crystals indicated by arrows. B. Flow through from the MACs magnet purification column containing predominantly merozoites with little hemozoin; a Geimsa-stained smear of this preparation confirmed the absence of hemozoin. C. Elute from the column containing predominantly hemozoin crystals. Preparations were stained with ethidium bromide (EtBr) and analysed by flow cytometry by size (side and forward scatter (SSC and FSC) and fluorescence (GFP and EtBr). After magnet purification, hemozoin crystals were removed.

FIG. 21 shows the relationship between merozoite invasion rate and the concentration of merozoites and RBCs. A. Purified merozoites and RBC were incubated at different concentrations of merozoites and RBCs. At a fixed concentration of RBCS, increasing the concentration of merozoites leads to a decreasing invasion rate as the merozoite:RBC ratio increases. At a fixed concentration of merozoites, increasing the concentration of RBCs leads to an increasing invasion rate as the merozoite:RBC ratio decreases. B. Increasing the merozoite:RBC ratio leads to increasing parasitemia, although the invasion rate decreases. The ‘invasion rate’ represents the proportion of merozoites that invaded RBCs.

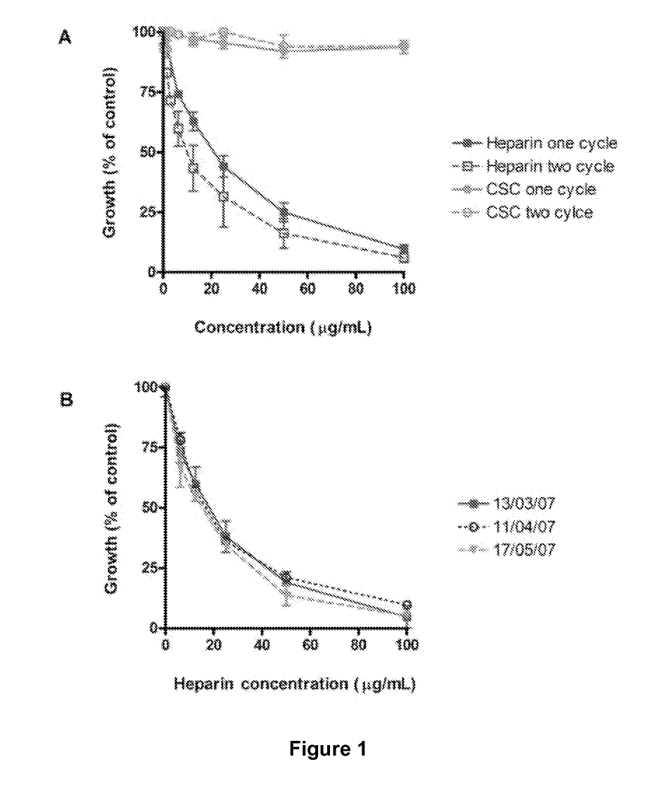

### DETAILED DESCRIPTION OF THE INVENTION

The present invention, is predicated at least in part on findings by the Applicant directed to structural and functional aspects of sulfated polysaccharide molecules that are necessary for, or involved with, inhibition of growth of P. falciparum. These findings may be applied to the screening of known sulfated polysaccharides for antiplasmodial activity, for the modification of polysaccharides or sulfated polysaccharides having known antiplasmodial activity to enhance that activity, and also to the de novo design of sulfated polysaccharide antiplasmodial agents, or any combination of screening, modifying or designing such agents. The identification of structure/function relationships also provides for new methods of treatment and prevention of Plasmodium infection using novel agents identified, produced or designed in accordance with the present invention. These relationships also provide for the new uses of known sulfated polysaccharide molecules in methods of treatment or prevention of Plasmodium infection.

These findings may also be applied to identify candidate antiplasmodial agents that act by binding to a plasmodial merozoite protein and interfering with the binding of said merozoite protein with heparin or derivative thereof. In one form of the invention the merozoite protein is merozoite surface protein 1 (MSP1), merozoite surface protein 2 (MSP2), Pf12, Pf38, Pf41 or apical membrane antigen 1. Preferably, the merozoite protein is MSP1. Where a sulfated polysaccharide antiplasmodial agent has undesired anticoagulant activity (for example, where the agent is heparin-based), the present invention further provides structure/function information allowing the reduction of anti-coagulant activity in the agent while maintaining antiplasmodial activity. It is proposed that sulfated polysaccharides produced or identified by the present invention will be useful in methods of treating and preventing malaria and other infections with Plasmodium spp.

In accordance with the above, the present invention provides in a first aspect a method for identifying antiplasmodial activity in a candidate sulfated polysaccharide molecule, the polysaccharide molecule comprising two or more disaccharide units, the method comprising one or more of the following steps: (i) assessing the average degree of sulfation per disaccharide unit, (ii) assessing the position of two or more sulfate groups in at least one of the disaccharide units, (iii) assessing the linkage of one or more sulfate groups, in at least one of the disaccharide units, (iv) assessing the saccharide backbone composition, wherein the candidate molecule. is considered to possess antiplasmodial activity if one or more of the following conditions is satisfied: (a) the average degree of sulfation is at least about 1 sulfate group per disaccharide unit, (b) 2 or more sulfate groups are present on a single monosaccharide residue of the disaccharide unit, (c) 50% or more of sulfate groups are O-linked, (d) the saccharide backbone composition comprises 50% or less of iduronic acid.

Thus, the candidate molecule is considered to possess antiplasmodial activity if one or more of the following conditions is satisfied: (a) the average degree of sulfation is at least about 1 sulfate group per disaccharide unit (b) 2 or more sulfate groups are present on a single monosaccharide residue of the disaccharide unit (c) 50% or more of sulfate groups are O-linked (d) the saccharide backbone composition comprises 50% or less of iduronic acid. Preferably, conditions (a), (b) and (c) are satisfied and most preferably conditions, (a), (b), (c) and (d) are satisfied.

In the context of the present invention, the term “sulfated polysaccharide” includes any sulfated polymeric carbohydrate formed of repeating sugar units, the sugar units joined by glycosidic bonds. The repeating units may be the same (a homopolysaccharide) or different (a heteropolysaccharide). The molecule may be linear or branched. More specifically, the present invention is concerned with polysaccharides formed of repeating disaccharide units.

The term “sulfated” in the context of the term “sulfated polysaccharide” relates to the addition of a sulfate group by means of a covalent bonding to any competent atom in the polysaccharide molecule.

In the screening method described above, the candidate sulfated polysaccharide molecule may be naturally occurring, or a derivative of a naturally occurring sulfated polysaccharide. The molecule may also be completely or partially synthetic. It will be understood that for the purposes of the inventive method it is not necessary for the candidate molecule to be in physical existence as a chemical compound. It is anticipated that the methods are also fully operable when only the chemical structure, or other relevant information (such as nuclear magnetic resonance data or X-ray crystallography data) are used to assess the presence and position of sulfation on the sulfated polysaccharide.

Preferred sulfated polysaccharide molelcules useful as candidate molecules include anionic oligo- and polysaccharides such as glycoaminoglycans (GAGs). This class of compound includes molecules such as chondroitin sulfate, keratin sulfate, heparin, dermatan sulfate and hyaluronate. GAGs are carbohydrates based on repeating units of uronic acid (uronate) (glucuronic acid (GlcA) or iduronic acid (IdoA)) and amino sugar residues (hexosamine) (N-acetyl glucosamine (GlcNAc) or N-acetylgalactosamine (GalNAc)). The family includes heparin and the closely related heparan sulfate (HS), as well as hyaluronic acid, chondroitin sulfate (CS), dermatan sulfate (chondroitin sulfate B) and keratan sulfate. During polymerization, these molecules undergo modification with epimerization of glucuronic acid to iduronic acid, and the addition of sulfation residues to various positions. GAGs are ubiquitously expressed throughout biological systems often as protein conjugates known as proteoglycans (PG) and are able to interact with a diverse set of proteins and other molecules eliciting a wide range of functions including cell signaling, migration and adhesion (reviewed [14]). 14. Kjellen L, Lindahl U (1991) Proteoglycans: structures and interactions. Annu Rev Biochem 60: 443-475.

In one embodiment of the Method, the sulfated polysaccharide is a heparin or derivative thereof. Heparin is based on repeating units of GlcA and GlcNAc and is the most extensively modified GAG. Heparin contains high levels of IdoA, extensive sulfation of GlcN at the amino residue (N-S) and the carbon 6 oxygen residue (6-Osulfation) and also sulfation of the uronic acid at carbon 2 oxygen residue (2-Osulfation). Other rarer sulfation substitutions are possible. The resulting compound is highly negatively charged with 2.4-2.7 sulfate groups/disaccharide unit (SO3-/COO ratio) (reviewed in [15,16]. Heparin, along with low molecular weight heparin and heparin pentasaccharide fondaparinux are already extensively used in the clinic as anticoagulants. Derivatives of heparin and related compounds (especially those with reduced anticoagulation properties), are typically considered to be safe for human use. 15. Capila I, Linhardt R J (2002) Heparin-protein interactions. Angew Chem Int Ed Engl 41: 391-412.16. Rabenstein D L (2002) Heparin and heparan sulfate: structure and function. Nat Prod Rep 19: 312-331.

Heparin is normally confined to intracellular compartments of mast cells where it is thought to be essential in the storage of granule proteases [17]. Heparan sulfate, is widely expressed as a proteoglycan (HSPG) found on many if not all cell surfaces. HSPG is involved in a variety of biological functions, including cell signaling, adhesion, migration, and proliferation (reviewed in [16,18]). HSPG has the same backbone composition as heparin, however undergoes less modification and is characterized by ‘heparin’ like regions, inter-dispersed with unmodified sections. 17. Humphries D E, Wong G W, Friend D S, Gurish M F, Qiu W T, et al. (1999) Heparin is essential for the storage of specific granule proteases in mast cells. Nature 400: 769-772.18. Jackson R L, Busch S J, Cardin A D (1991) Glycosaminoglycans: molecular properties, protein interactions, and role in physiological processes. Physiol Rev 71: 481-539.

Fondaparinux is a synthetic carbohydrate based on the minimal sequence required for binding and activation of antithrombin III (ATIII), with a stabilizing methyl group at the reducing end of one of the monosaccharide units. The IC50 of Fondaparinux against Plasmodium falciparum invasion is approximately 100 ug/ml. Fondaparinux may be modified to reduce anticoagulation activity and/or increase merozoite invasion inhibition activity and in one form, in accordance with the invention.

In one form, the method requires an assessment of the average degree of sulfation (d.o.s). As the skilled person will understand, a preparation of polysaccharide molecules may be heterogenous with respect to parameters such as length, and the number and position of sulfate groups attached to the polysaccharide backbone. Thus, the method requires that an average d.o.s across different molelcules in a preparation is calculated. Where there is no heterogeneity, the average d.o.s will be the absolute d.o.s. Furthermore, gaps in sulfation are sometimes noted along the length of a sulfated polysaccharide molecule. Therefore an average d.o.s. may also be required where the level of sulfation is not uniform across a single sulfated polysaccharide molecule. For example, one segment of the molecule may be relatively highly sulfated, while another may be relatively lowly sulfated but the molecule as a whole has an average d.o.s. between those two sulfation levels. Where the level of sulfation is uniform across a single molecule, the average d.o.s. will be the absolute d.o.s

For the purposes of the present method, the d.o.s may be calculated by reference to the number of sulfate groups bound to a single disaccharide repeat unit.

Antiplasmodial activity is indicated where the average d.o.s is at least 1 sulfate group per disaccharide unit. In another form of the method, activity is indicated where the average d.o.s is at least 1.5 sulfate groups per disaccharide unit. As an example, this situation may occur where the sulfated polysaccharide molecule is composed of 3 disaccharide units, and the first and third disaccharide units are each mono-sulfated, with the second being unsulfated.

The skilled person appreciates that the level of sulfation may assessed by reference to weight, or by disaccharide composition analysis. It will be understood that such other measures are easily convertible to d.o.s, and such measures are included within the scope of this invention.

The method may further require an assessment of the position of two or more sulfate groups in at least one of the disaccharide units. In the context of the present invention, the term “position” relates to whether or not two or more sulfate groups are bonded to the same monomer within the disaccharide unit. For example, where the sulfated polysaccharide is composed of 3 disaccharide units (i.e. 6 monomers), and the first monomer exhibits 2 sulfate groups, this molecule would be considered to have antiplasmodial activity according to the present method. By contrast, a sulfated polysaccharide molecule having a first sulfate group on the first monomeric unit and a second sulfate group on the third monomeric unit would be considered to lack putative antiplasmodial activity.

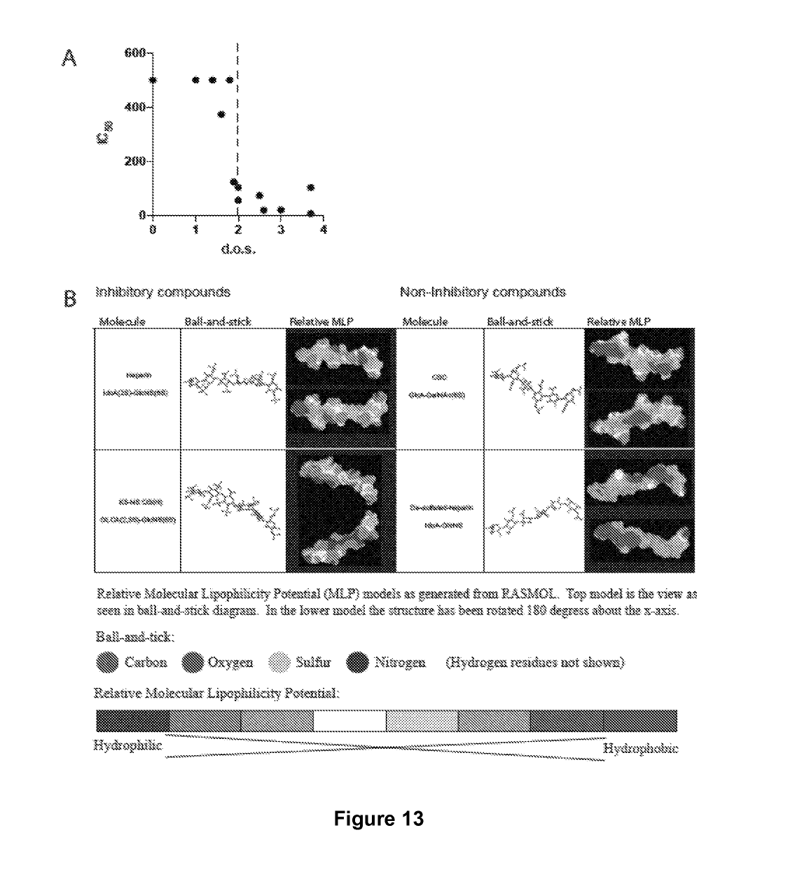

In support of the relevance of the presence and specified positioning of sulfate groups, a carbohydrate modeling program (Sweet II) was used to generate 3D structural models of inhibitory and non-inhibitory molecules, and the relative molecular lipophilicity potential of the surface was predicted to further understand the basis of inhibitory activity (FIG. 13B). Structures were modeled as 6 mers, and were based on the most abundant disaccharide unit of each compound. Comparison of representative models of inhibitory and non-inhibitory compounds showed that different sulfation patterns and levels result in differences in the extents and patterns of hydrophobic and hydrophilic surfaces that may be involved in interactions. All inhibitory molecules appeared to contain hydrophilic ridges seen in heparin and K5-NSOS-H and a higher level of sulfation corresponds with a larger hydrophilic surface. This corresponds with the need for an average degree of sulfation of greater than or equal to 1.5 (d.o.s. 1.5) (FIG. 13A). Non-inhibitory compounds appeared to have predominantly hydrophobic regions as seen with de-N,O-sulfated heparin and CSC. Without wishing to be limited by theory in any way, it is proposed that although the hydrophilic surface of inhibitory molecules appears relevant, the correct spatial arrangement of sulfate groups on inhibitory molecules may be needed for optimal binding to their targets.

In other embodiments of the method, the average d.o.s is at least about 2, 2.5, 3, 3.5, 4, 4.5 or 5 sulfate groups per disaccharide unit.

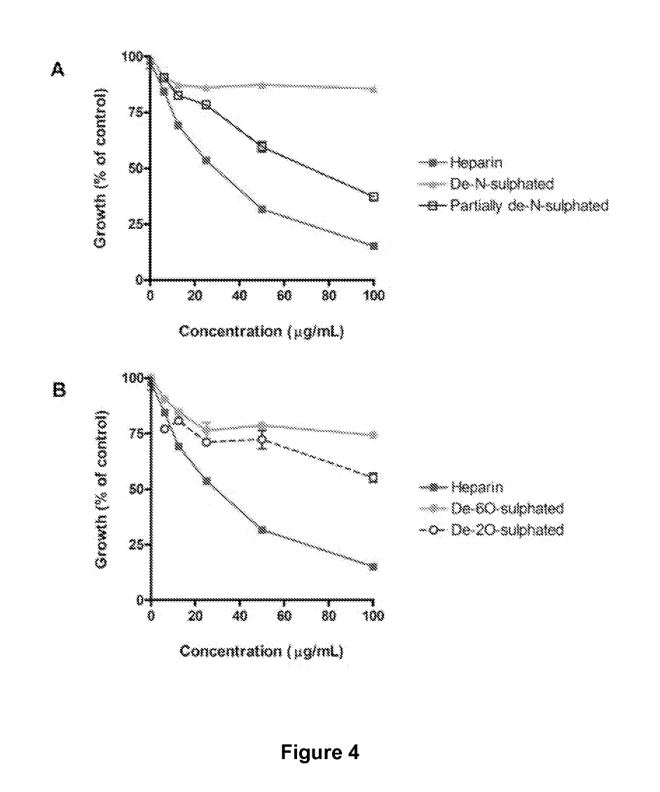

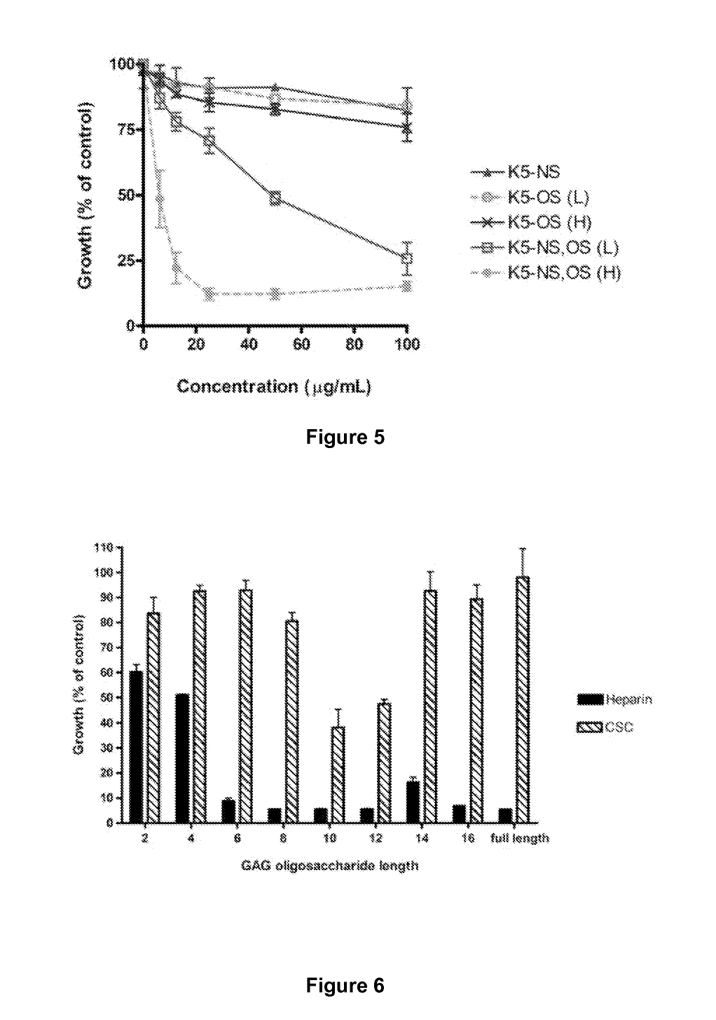

The importance of a minimum degree of sulfation has also been demonstrated using capsular polysaccharide from E. Coli K5 as a model sulfated polysaccharide. It has been previously presumed that the substantial negative charge of sulfated polysaccharides such as heparin is responsible for the anti-infective activities of these molecules. To establish that the inhibitory activity of heparin and heparin-like compounds is not due solely to negative charge (FIG. 13 A, Table 2) and to investigate structure/function activity of heparin like compounds, a number of modified heparin compounds, chemically modified K5 derivatives and chondroitin sulfate compounds (CS) were tested by the Applicant for inhibition of P. falciparum blood stage growth (Table 1, Table 2, FIG. 10). K5 derivatives tested were: de-N and O-sulfated heparin, partially and completely de-N-sulfated heparin, de-6O-sulfated heparin and de-2O-sulfated heparin (FIG. 4A, Table 1A, Table 2), K5 polysaccharide N sulfated (100%, K5-NS), low level O sulfated (90% 6-O sulfated GlcNAc, 10% 2-O sulfated GlcA, K5-OS-L), high level O sulfated (100% 6-O sulfated and unknown 3-O sulfated GlcNAc, 100% 2-O,3-O di-sulfated GlcA, K5-OS-H), low level N and O sulfated (100% N sulfated, 90% 6-O sulfated GlcN, 10% 2-O sulfated GlcA, K5-NS,OS-L), and high level N and O sulfated (100% N, 100% 6-O sulfated GlcN, 70% 2O,3-O di-sulfated, 30% 2-O or 3-O singular sulfated GlcA, K5-NS,OS-H) (FIGS. 4B and C, Table 1B, Table 2,). CS compounds tested where, CSC (10% GalNAc-4S, 88% GalNAc-6S), CSD (46% GalNAc-6S, 28% GlcA-2S, 23% GlcA-2S-GalNAc-6s), CSE (21% GalNAc-4S, 10% GalNAc-6S, 60% GalNAc-4S,6S), CSB-2,6S (21% GalNAc-6S, 76% IdoA-2S-GalNAc-6S) and CSB-4,6S (28% GalNAc-4S, 66% IdoA-2S-GalNAc-4S) (FIG. 5, Table 1C, Table 2B). These results further demonstrate that disulfation of amino sugar residues may increase antiplasmodial activity.

In addition to the above studies and to further discount the potential for heparin to be acting solely due to net negative charge Applicant analyzed correlation of activity of compounds with d.o.s. to demonstrate associations between activity and level of sulfation (FIG. 13A). This analysis shows that activity may require a threshold level of sulfation of approximately d.o.s.≧1.5. It is notable that both K5-NSOS-H and EK-NSOS-H have the same high negative charge (d.o.s.=3.7) but have very different inhibitory activity (IC₅₀ 7.4 and 103 μg/mL), while CSE has a comparable low d.o.s. of 1.5 but IC₅₀ of 26.7 μg/mL.

In addition to the above studies, modifications to Fondaparinux are envisioned. Without wishing to be bound by theory, for example, 3-O-sulfation of the middle glucosamine group that is essential for anticoagulant activity may be removed. A further modification is the replacement of IdoA with GlcA; as discussed herein, GlcA has higher inhibitory activity against Plasmodium falciparum merozoite invasion. Furthermore, the d.o.s. may be increased by addition of 2 and/or 3 O-sulfation to uronic acid residues.

The term “antiplasmodial activity” includes any biological activity that inhibits, prevents, delays or otherwise interferes with a biological process required for the reproduction, growth, viability or transmission of a Plasmodium spp, measured in vivo or in vitro, or using a wild type or laboratory strain of the parasite. The lifecycle of Plasmodium falciparum commences when haploid sporozoites are injected into the human host by an infected female Anopheles mosquito taking a blood meal. The sporozoites invade hepatocytes in the liver and undergo schizogony (asexual division), resulting in the production of large numbers of merozoites. These merozoites subsequently invade red blood cells and undergo maturation within the erythrocyte through the ring, trophozoite and schizont stages. Merozoites are then released upon rupture of the erythrocyte to reinvade other, uninfected, red blood cells. This asexual replication may then be repeated or sexual differentiation may occur to form immature macro- (female) and micro-gametocytes (male). The exoerythrocytic mature macro- and micro-gametocytes are taken up by a feeding mosquito, signifying the beginning of the sexual lifecycle within the mosquito. Within the mosquito, the microgametocyte undergoes rapid DNA replication followed by cell division, resulting in the formation of flagellated cells: These flagellated cells invade the macrogametocyte to form the diploid zygote that subsequently undergoes meiosis and develops into the ookinete. The ookinete embeds itself in the mosquito midgut wall, becoming an oocyst that undergoes sporogony resulting in the production of large numbers of haploid sporozoites that migrate to the salivary glands of the mosquito. Sporozoites are then injected into a new human host during feeding of the mosquito vector. Accordingly, biological processes include parasite attachment, schizont rupture, merozoite invasion of an erythrocyte, and ring formation. Antiplasmodial activity may be conveniently measured by using any one of the many model systems known to the skilled person, including those described herein. Typically, replicate parasite cultures are exposed to increasing levels of the antiplasmodial agent and an assessment is made as to growth of the parasite as a proportion of growth exhibited in a control culture. It will be understood that an agent will be considered to have antiplasmodial activity if the agent fails to completely inhibit growth of the parasite. Even agents showing only a low level of activity (for example, capable of decreasing growth by 5% as compared with a control molecule or vehicle) are still considered to demonstrate antiplasmodial activity.

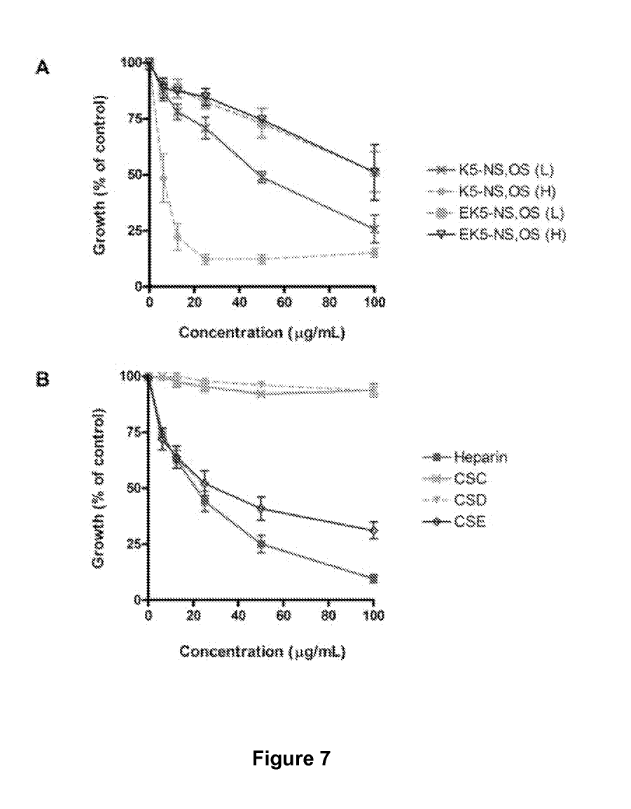

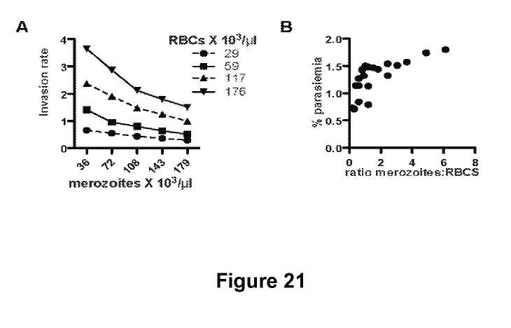

The experimental work described herein has clearly identified the cellular mechanism of antiplasmodial activity of sulfated polysaccharides such as heparin, showing inhibition in the early steps in the merozoite invasion of erythrocytes (FIG. 7 and FIGS. 14 to 21). Previously, it has been reported that heparin inhibits via blocking of schizont rupture [19,20], and although Applicant has demonstrated some delay in schizont rupture in the presence of heparin, this delay may not be sufficient to explain the dramatic reduction in ring formation (FIG. 7 A and B). Although P. falciparum blood stage replication occurs over a 48 hour period, the time from schizont rupture to merozoite invasion and ring formation is highly dynamic, occurring in a 1-5 minute time period. 19. Butcher G A, Parish C R, Cowden W B (1988) Inhibition of growth in vitro of Plasmodium falciparum by complex polysaccharides. Trans R Soc Trop Med Hyg 82: 558-559.20. Evans S G, Morrison D, Kaneko Y, Havlik I (1998) The effect of curdlan sulphate on development in vitro of Plasmodium falciparum. Trans R Soc Trop Med Hyg 92: 87-89.

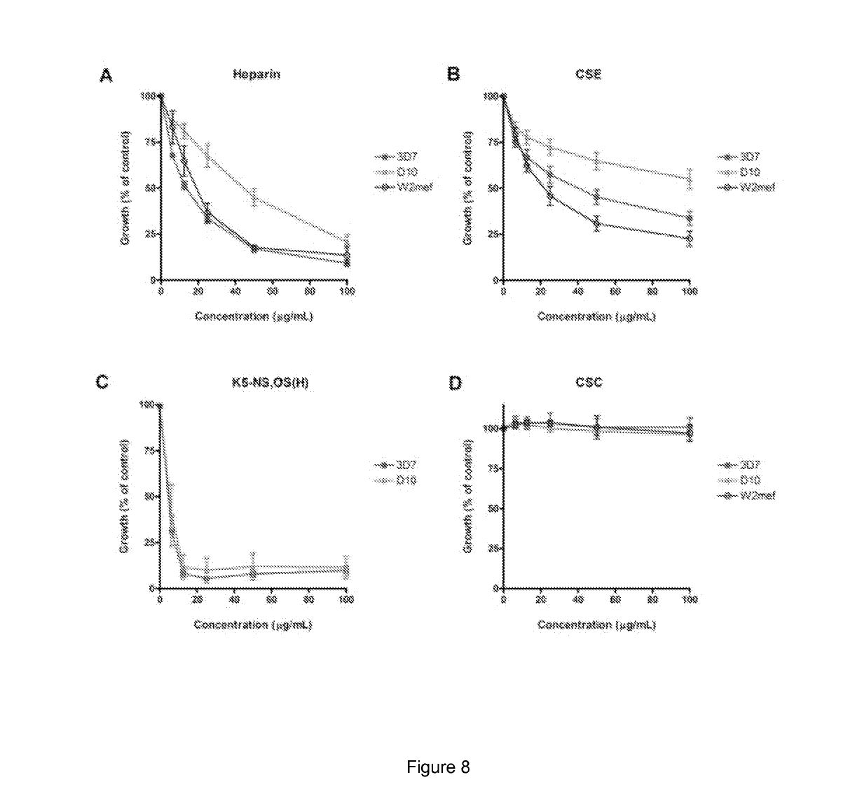

Without wishing to be limited by theory, the antiplasmodial activity at initial contact events appears to be via a mechanism of merozoite invasion, with multiple parasite lines showing comparable inhibition by heparin. Different P. falciparum laboratory and wild isolates are known to use multiple invasion pathways, utilizing a variety of merozoite proteins and erythrocyte receptors. This is thought to be one mechanism of immune invasion employed by P. falciparum. One major alternative pathway identified is the selective use of sialic acid receptors. Understanding of the use of different pathways has been shown with differing invasion phenotypes into enzymatic treated erythrocytes. It is demonstrated herein that sulfated polysaccharides such as heparin appear to inhibit multiple identified invasion pathways to the same degree. 3D7 (SA independent) and W2mef (SA dependent) and three other genetically distinct parasite lines were all inhibited to similar levels by heparin, as well as other inhibitory compounds CSE and K5-NSOS-H (FIG. 6, FIG. 8A). Furthermore, inhibition of 3D7 into neauraminidase, trypsin and chymotrypsin erythrocytes was comparable normal invasion (FIG. 8B). This suggests that heparin inhibits a step of erythrocyte invasion. The identification of this mechanism may lead to identification of a target of vaccine and drug development.

In one form, the method requires an assessment of the linkage of one or more sulfate groups in at least one of the disaccharide units, with antiplasmodial activity being indicated where the 50% or more of sulfate groups are O-linked. In previous art it has been proposed that antiplasmodial activity in a sulfated polysaccharide molecule is dependent on N and O sulfate groups. However, here the Applicant shows herein that N and O sulfation are not absolute requirements with two sulfate groups linked at either N or O positions on a single monomeric unit of a disaccharide sufficient for activity.

In one form of the method the presence of two sulfate groups on a disaccharide unit and d.o.s. ≧1.5 is particularly indicative of antiplasmodial activity. A number of compounds having known antiplasmodial activity have been shovi to possess these attributes, for example heparin, partially de-N-sulfated heparin, K5-OS-H, K5-NSOS-L, K5-NSOS-H and CSE. Antiplasmodial activity is dependent on spatial positioning of sulfate groups and backbone residues and not only d.o.s. with compounds with high d.o.s. being non-inhibitory in some cases (for example EK5-NSOS-L and EK5-NSOS-H).

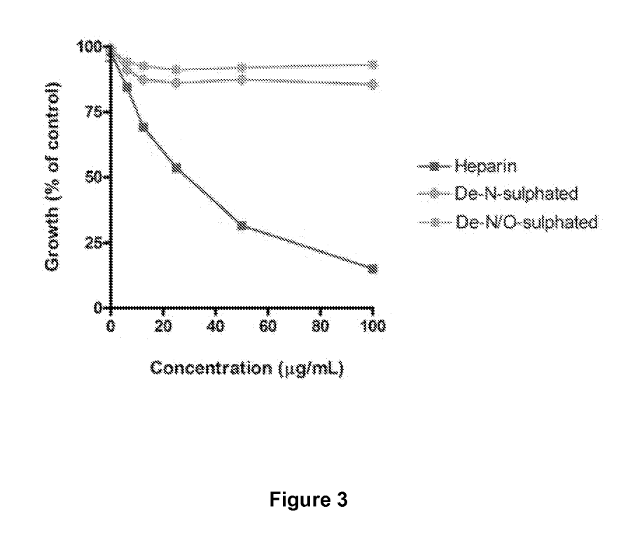

The importance of sulfate groups in inhibitory activity of the sulfated polysaccharide heparin was investigated with a number of modified heparin compounds (Table 1a). De-N-sulfation of glucosamine residues (de-N-sulfated), or de-N and de-O-sulfation of glucosamine and uronic acid residues, (de-N/O-sulfated) resulted in the loss of inhibitory activity against P. falciparum (FIG. 3). This shows that the presence of sulfation groups is required for inhibitory activity. Inhibitory activity of fully de-N-sulfated heparin and partially de-N-sulfated heparin showed reduction of N-sulfation decreased inhibitory activity (Table 2, FIG. 4a) demonstrating that inhibitory activity of heparin is at least partially dependent on the level of N-sulfation, and particularly the presence of di-sulfation of a single disaccharide unit.

Inhibitory activity of heparin was reduced with the selective de-sulfation from oxygen residues: de-6-O-sulfated or de-2-O-sulfated (FIG. 4b). De-2-O-sulfated heparin had significantly greater inhibitory activity than de-6-O-sulfated heparin (IC₅₀ 123 and 373 μg/mL respectively) (Table 2). These data show that O-sulfation residues are required for the inhibitory activity of heparin, and suggests 6-O-sulfation of glucosamine has a greater role than 2-O-sulfation of uronic acid again due to the requirement of di-sulfation of a single monomeric unit.

It is further demonstrated herein that the length of the sulfated polysaccharide molecule may be a determinator of antiplasmodial efficacy (FIG. 4D). In a further embodiment the molecule is considered as having antiplasmodial activity where it comprises at least 3 disaccharide units. Yet a further embodiment provides that the sulfated polysaccharide molecule is considered as having antiplasmodial activity if it consists of at least 4 disaccharide units. Another embodiment provides that the sulfated polysaccharide is considered as having antiplasmodial activity if it comprises at least 5 disaccharide units. Another embodiment provides that the sulfated polysaccharide is considered as having antiplasmodial activity if it comprises at least 6 disaccharide units. Another embodiment provides that the sulfated polysaccharide is considered as having antiplasmodial activity if it comprises at least 7 disaccharide units. Another embodiment provides that the sulfated polysaccharide is considered as having antiplasmodial activity if it comprises at least 8 disaccharide units. Another embodiment provides that the sulfated polysaccharide is considered as having antiplasmodial activity if it comprises at least 9 or 10 disaccharide units. Another embodiment provides that the sulfated polysaccharide is considered as having antiplasmodial activity if it comprises at least 20, at least 50, at least 100, at least 500, at least 1000 or at least 10,000 disaccharide units. While molecules in excess of 8 disaccharide units may exhibit antiplasmodial activity, it is proposed that molecules having 6 or 8 disaccharide units may have optimal efficacy.

It will be understood that antiplasmodial activity is not contraindicated where the sulfated polysaccharide molecule consists of a less than whole number: For example, a molecule consisting of 2.5 disaccharide units (i.e. 5 monosaccharide residues) is not defined by the present methods as lacking antiplasmodial activity on the basis of that consideration alone.

By reference to FIG. 4D Applicant has demonstrated that for heparin, the full length molecule is not required for antiplasmodial activity, with molecules as short as 4 sugar residues being efficacious in vitro. Where a candidate agent shows efficacy as an antiplasrnodial, it may be possible to truncate the chain to provide a shorter molecule that still retains efficacy. A shorter chain may provide certain advantages, such as greater stability in the gut (for oral medicaments), lower immunogenicity, or decreased anticoagulant activity, or reduced difficulty and cost in synthesis of compound.

The identity of the disaccharide units from which the polysaccharide is composed is further indicative of antiplasmodial efficacy. Accordingly, in one embodiment, the method includes the step of assessing the identity of at least one residue of the polysaccharide molecule. A molecule may be considered to have antiplasmodial activity if one or more of the residues is uronic acid or hexosamine. Analysis of inhibitory sulfated polysaccharide molecules suggests enhanced activity where either the uronate or hexosamine residues within a disaccharide unit are di-sulfated. The importance of uronic acid composition (glucuronic acid (GlcA) compared to epimerised iduronic acid (IdoA)) in inhibitory activity was assessed by testing epimerized K5 (EK5) derivatives for inhibition of P. falciparum blood stage growth in GlAs. EK5-NS,OS-L and EK-NSOS-H are prepared from K5-NSOS-L and K5-NSOS-H, and are thought to contain 50:50 IdoA to GlcA ratio in alternating pattern along the carbohydrate chain (Table 1b). Both epimerised K5 compounds showed significantly lowered inhibitory activity compared to parent molecules (FIG. 4C Table 2). Thus, neither epimerization of uronic acid, nor a mixture of GlcA and IdoA is contraindicated in a candidate molecule.

The importance of amino sugar residues, glucosamine (GlcN) compared to galactosamine (GalN), for inhibitory activity was investigated by testing three chondroitin sulfate (CS) molecules for inhibition of P. falciparum blood stage growth. CSC, CSD, and CSB-2,4S were not inhibitory, while CSB-2,6S showed some inhibitory activity CSE showed substantial inhibitory activity (IC₅₀ 26.7 μg/mL) (Table 2, FIG. 5). The inhibitory activity of CSE suggests that GlcN is may not be required for the inhibitory activity of heparin and that sulfate groups determine activity of compounds.

In one embodiment of the method the sulfated polysaccharide is composed of less than 50% iduronic acid. Without wishing to be limited by theory in any way, it is proposed that reducing iduronic acid content may be beneficial in limiting or avoiding deleterious alteration to normal coagulation in a recipient subject. In particular, it is proposed that a reduction in IdoA in a sulfated polysaccharide molecule may avoid the problem of unwanted bleeding. In one embodiment, the sulfated polysaccharide molecule is composed of less than about 40, 30, 20, or 10% iduronic acid. In another embodiment the molecule is composed of about 0% iduronic acid.

Another aspect of the present invention provides a sulfated polysaccharide molecule identified by a screening method as described herein.

As will be appreciated from the foregoing, Applicant has provided valuable guidance in the assessment of sulfated polysaccharides as potential antiplasmodial agents. It will be understood that these findings are also applicable to methods for producing an antiplasmodial agent. Accordingly, in a further aspect the present invention provides a method for producing or rationally designing a sulfated polysaccharide having antiplasmodial activity, the method comprising the steps of providing a polysaccharide molecule or a sulfated polysaccharide molecule having two or more disaccharide units, modifying the polysaccharide or sulfated polysaccharide molecule by one or more of the following methods, (i) alter or ensure the average degree of sulfation to about 1 sulfate group per disaccharide unit, (ii) alter or ensure the position of sulfation such that 2 or more sulfate groups are present on a disaccharide unit, (iii) alter or ensure the linkage of sulfation such that 50% or more of sulfate groups are O-linked, (iv) alter or ensure that the saccharide backbone composition comprises 50% or less of iduronic acid.

The starting position (whether a physical chemical compound or a virtual chemical structure) for such methods may be any sulfated polyskcharide already known to exhibit antiplasmodial activity with a view to improving efficacy. Direction regarding the position, level and linkage of sulfate groups; and the identity of carbohydrate residues and length of the overall molecule are all applicable to methods for producing an efficacious polysaccharide molecule. A number of methods are available to modify heparin and heparin like compounds, including K5 polysaccharides, including fractionation using heparinase enzymes and enzymatic modification with the addition of sulfate groups (for example [21] or [22]). Advances in gylcan biology have made the chemical and enzymatical synthesis of discrete known oligosaccharide sequences achievable [23]. These advances will continue, over coming some of the difficulties and cost restrictions of synthesis of glycoasaminog lycans [24]. 21. Casu B, Grazioli G, Razi N, Guerrini M, Naggi A, et al. (1994) Heparin-like compounds prepared by chemical modification of capsular polysaccharide from E. coli K5. Carbohydr Res 263: 271-284.22. Rusnati M, Oreste P, Zoppetti G, Presta M (2005) Biotechnological engineering of heparin/heparan sulphate: a novel area of multi-target drug discovery. Curr Pharm Des 11: 2489-2499.23. Blow N (2009) Glycobiology: A spoonful of sugar. Nature 457: 617-620.24. Linhardt R J, Dordick J S, Deangelis P L, Liu J (2007) Enzymatic synthesis of glycosaminoglycan heparin. Semin Thromb Hemost 33: 453-465.

It is possible that the method may utilize fragments of polysaccharides or sulfated polysaccharides (even down to the level of monosaccharide or sulfated monosaccharide) as a starting point in which case the method may be preceded commence with the polymerization of the fragments of polysaccharides or sulfated polysaccharides.

In a further aspect, the present invention provides a sulfated polysaccharide molecule identified, produced or rationally designed according to a method described herein.

As will be apparent to the skilled artisan, sulfated polysaccharide molecules identified, or produced, or rationally designed according to the present invention may be useful as pharmaceutical agents for the treatment of animals, and particularly humans for infection with Plasmodium spp. Accordingly, the present invention further provides a composition comprising a sulfated polysaccharide agent as identified, produced or rationally designed as described by a method herein, and a pharmaceutically acceptable carrier.

The sulfated polysaccharides for human use may have a molecular weight of 1,000-1,000,000, preferably 5,000-500,000, more preferably 20,000-200,000, especially 50,000-120,000 as an average molecular weight measured by gel permeation chromatography. For clinical use, it is desirable for the sulfated polysaccharide to be purified to remove or limit the heterogeneity often inherent in preparations of these molecules. For example, methods to decrease heterogeneity of sulfated polysaccharide preparations include: mass spectrometry, gel filtration, HPLC, anion ion exchange chromatography and enzymatic treatment of sulfated polysaccharides (see for example, Chai et al, J Biol, Chem 2002, 277:22438).

Sulfated polysaccharides are preferably formulated and administered in the form of pharmaceutically acceptable salts. Preferred salts are alkali metal salts or alkali earth metal salts (sodium salts, potassium salts, calcium salts and magnesium salts, etc.), and ammonium salts or nontoxic amine salts (ammonium salts, tetramethylammonium salts, tetraethylammonium salts, methylammonium salts, dimethylammonium salts, trimethylammonium salts, triethylammonium salts, diethylammonium salts, ethylammonium salts, lysine salts, arginine salts, ethylenediamine salts, ethanolamine salts, diethanolamine salts, piperidine salts and piperazine salts).

The prior art provides sufficient teaching to allow the skilled person to formulate sulfated polysaccharides as pharmaceutical compositions comprising a sulfated polysaccharide molecule as described herein and a pharmaceutically acceptable carrier. Pharmaceutical compositions of the present invention contain a pharmaceutically acceptable carrier. The pharmaceutically acceptable carrier will typically depend at least in part on the dosage form. When the pharmaceutical compositions are used for oral administration, they may appropriately contain pharmaceutically acceptable carriers including binders such as gum tragacanth, gum arabic, corn starch and gelatin; excipients such as dicalcium phosphate; disintegrants such as potato starch and alginic acid; lubricants such as magnesium stearate; sweetening agents such as sucrose; dyes; and perfumes such as orange flavor; and solvents such as water, ethanol and glycerol.

Where pharmaceutical compositions of the present invention are injectable compositions, suitable pharmaceutically acceptable carriers include sterilized water, isotonic saline and pH buffers. Alternatively, injectable compositions of the present invention may be sterilized powder compositions or lyophilized powder compositions that can be used by simple dissolution in sterilized water. Injectable pharmaceutical compositions of the present invention may contain sugars (glucose, mannitol and dextran, etc.), polyhydric alcohols (glycerol, etc.), and inorganic salts (sodium salts and magnesium salts, etc.).

When pharmaceutical compositions of the present invention are administered by intravenous infusion, they may contain nutrients such as glucose, vitamins, amino acids and lipids.

Pharmaceutical carriers to be added to dosage forms for other administration modes such as nasal administration, inhalation and transdermal administration are also well-known to those skilled in the art.

Where pharmaceutical compositions of the present invention are orally administered, they may be in the form of controlled- or sustained-release formulations. Well-known sustained-release formulations include ordinary sustained- or controlled-release formulations such as gel-coated formulations and multicoated formulations as well as site-specific delivery formulations (e.g. burst release at pyloric regions or effervescent delivery to the duodenum). Oral compositions include, for example, tablets, pills, capsules, ampoules, sachets, elixirs, suspensions, syrups, etc.

The dosage forms and pharmaceutical carriers mentioned above, and other relevant formulations are described in Remington's Pharmaceutical Sciences, 19th ed., Mack Publishing Company, which is incorporated herein by reference.

A sulfated polysaccharide may be administered to a patient with Plasmodium infection as a pharmaceutical composition of the present invention in a unit dose of a pharmaceutical composition containing a sulfated polysaccharide. As used herein, the term “unit dose” includes not only individually packaged unit doses such as vials but also aliquots dispensed from vials into syringes and compositions for infusion contained in infusion containers.

The skilled person, will gain significant guidance from existing formulations of sulfated polysaccharides, such as the heparins. For example, the sulfated polysaccharide may be simply formulated for injection using benzyl alcohol (CAS 100-51-6, 1%) in pyrogen free water. Transdermal formulations are also contemplated using Phospholipon® 80 (PL80) and sphingomyelin for example. An oral formulation is further contemplated in which the agent is chemically conjugated with deoxycholic acid and DMSO molecules by secondary interactions. Another potential oral formulation uses glycyrrhetinic acid as permeation enhancer. Many other formulations will be operable, and all are included in the scope of this application.

In a further aspect the present invention further provides for methods for the treatment, and prevention of Plasmodium infection comprising the administration to a subject in need thereof an effective amount of composition described herein, wherein the sulfated polysaccharide is hot a compound selected from the group consisting of heparin, heparin sulfate, pentosan polysulfate, dextran sulfate, curdlan sulfate, cellulose sulfate, a carrageen and fucoidan.

Also provided is a method for treating or preventing Plasmodium invention, the method comprising the step of administering to a mammal in need thereof an effective amount of a sulfated polysaccharide molecule having one or more of the following structural features:(a) an average degree of sulfation of at least about 1 sulfate group per disaccharide unit (b) 2 or more sulfate groups present on a single monosaccharide residue of the disaccharide unit (c) the 50% or more of sulfate groups are O-linked (d) the saccharide backbone comprises 50% or less iduronic acid. Preferably, the sulfated polysaccharide molecule has all four features.

In the present invention, the effective amount of sulfated polysaccharide used for the treatment of malaria is typically 1-1,000 mg/kg weight daily, and preferably 5-500 mg/kg weight daily depending on the age, body weight and condition of the patient and the administration mode. Exemplary unit dosage compositions include a solution containing 100 mg of sulfated polysaccharide, 50 mg of mannitol, 18 mg of dibasic sodium phosphate and phosphate buffer (pH 6.5) per vial. Such a composition may be intravenously administered at 4 mg/kg every 8 hours in a total daily dose of 12 mg/kg for 4 days.

A person skilled in the clinical arts is enabled to arrive at an appropriate dosage given any particular sulfated polysaccharide. Where the toxicity of the sulfated polysaccharide is unknown, a preclinical toxicology study using an appropriate mammal (such as a mouse) may be undertaken to calculate an upper dosage for a human subject based on simple extrapolation. Typically, to establish an efficacious dosage the clinician commences treatment with a very low dosage, and titrates the dosage upwards until the desired clinical effect or clinical endpoint is noted. For example, the clinician may investigate the efficacy of the sulfated polysaccharide in curing primary blood stage infections (chemotherapeutic efficacy), or the efficacy in curing primary infections and in preventing secondary infections (composite chemotherapeutic and post-treatment prophylactic efficacy), or in reducing the post-treatment incidence of malaria and its complications (clinical risk reduction). One particular test of the chemotherapeutic efficacy of an antimalarial drug against primary malaria episodes can be estimated by the established “in vivo test” methodology (White N J (1997). Antimicrob Agents Chemother 41: 1413-1422). This test observes two key events. The first criterion is the alleviation of clinical symptoms and the suppression of the density of the pathogenic asexual blood stage parasites in the peripheral blood below the light-microscopic detection threshold (around 20-50 parasites/μl) within the first few days (avoiding “early treatment failure”). The second event is the potential recrudescence of persistent asexual blood stage parasites after one week (“late parasitological treatment failure”), which may or may not be associated with clinical symptoms of malaria (“late clinical treatment failure”).

One or more well known laboratory tests may be utilized to decide an efficacious dosage including: peripheral blood smear, Plasmodium antigen levels, and Plasmodium DNA levels (for example by quantitative PCR). The dosage regime may be further refined by reference to the same parameters. For example, it may be found that Plasmodium DNA levels can be kept to a minimum by dividing a daily dosage into 3 separate dosages taken at 8 hourly intervals.

The present invention provides sulfated polysaccharide molecules having one or more of structural features (i) to (iv) for use in the treatment and/or prevention of infection with Plasmodium. Further provided is a method for the prevention and/or treatment of a subject with, a Plasmodial infection comprising administering to said subject a sulfated polysaccharide molecule having one or more of structural features (i) to (iv). Also provided is the use of such molecules in the manufacture of a medicament for the prevention and/or treatment of a Plasmodium infection.

Indeed, a number of such molecules are disclosed herein, and include K5-OS-H, K5-NSOS-L, K5-NSOS-H, EK5-NSOS-L and EK5-NSOS-H, K5-OS-H, CSE, CSB-2,6,-OS, and fondaparinux.

In one embodiment of the invention, the sulfated polysaccharide is not heparin or a derivative thereof. Previous work, [8,9] has concentrated on heparin and derivatives. Prior art attempts to selectively de-sulfate specific residues are complicated by the de-sulfation of off-target positions. Furthermore, when using such molecules there are difficulties of distinguishing consequences of specific desulfation compared to consequences from loss of overall negative charge. These studies have overcome these limitations with the use of K5 derivatives with specific. sulfation modifications. Non-heparin based molecules are less likely to evoke undesirable anticoagulant activity in the recipient subject.

In one embodiment of the method the sulfated polysaccharide is an E. coli K5 capsular polysaccharide, or a derivative thereof. The structural features of heparin involved in the majority of heparin protein interactions remain ill-defined in part due to the difficulty of synthesizing heparin compounds with known structures. One method of studying heparin structure-function relationships is by using K5 polysaccharides. These carbohydrates are isolated from the capsule of Escherichia coli K5 [25] and have the same structure as unmodified heparin and heparan sulfate. K5 polysaccharides can be used as base molecules for the generation of semi-synthetic modified heparin-like compounds with variable levels and patterns of sulfation (K5 derivatives) [26]. 25. Vann W F, Schmidt M A, Jann B, Jann K (1981) The structure of the capsular polysaccharide (K5 antigen) of urinary-tract-infective Escherichia coli 010:K5:H4. A polymer similar to desulfo-heparin. Eur J Biochem 116: 359-364.26. Razi N, Feyzi E, Bjork I, Naggi. A, Casu B, et al. (1995) Structural and functional properties of heparin analogues obtained by chemical sulphation of Escherichia coli K5 capsular polysaccharide. Biochem J 309 (Pt 2): 465-472.

It is further accepted that at the filing date of this application, a number of sulfated polysaccharide molecules having one or more of structural features (i) to (iii) were known to be useful in the treatment or prevention of infection with Plasmodium, and it is intended that such compounds are excluded from the methods of treatment and prevention as described herein. Such prior art compounds include heparin, heparin sulfate, pentosan polysulfate, dextran sulfate, curdlan sulfate and fucoidan. It is important to note that while certain sulfated polysaccharides were known to be efficacious, the structural basis for that efficacy was not known at the filing date of this application. As such, while the antiplasmodial properties of a sulfated polysaccharide such as heparin may have been appreciated at the filing date of this application, the skilled person was not aware of the relevance of structural features (i), (ii) or (iii), and thus could not identify other molecules on the basis of those features.

While the methods of treatment and prevention are directed mainly to malaria caused by infection with Plasmodium falciparum, the methods also extend to the treatment of infection with Plasmodium malariae, Plasmodium ovale, Plasmodium vivax. and Plasmodium knowlesi.

The structure/function studies of sulfated polysaccharides detailed in this specification have further applicability to the assessment of anticoagulant activity of the polysaccharide. Prior art molecules that are known to be active against Plasmodium spp (such as heparin) may also lead to bleeding disorders in a recipient. Prior art has well established structure/function relationship of heparin as an anticogulant with a defined pentasaccharide containing an essential 3-O-sulfation residue required for activity [27]. Furthermore, it is known that K5 sulfated derivatives with predominantly GlcA, such as K5-NSOS-H, have low anticoagulation activity [22,28]. Also relevant to this point, anticoagulation activity may be lowered by the avoidance of IdoA in the sulfated polysaccharides of the present invention. Prior art, coupled with the knowledge of structure/function activity of heparin like compounds as antiplasmodial agents demonstrated here will enable the design of antiplasmodial agents lacking anticoagulation activity. Once an antiplasmodial property is identified, the anticoagulation property may be investigated using by any one of the known methods for measuring anticoagulation activity. Additionally, the agent may be modified to decrease anticoagulation activity by decreasing the level of 3-O-sulfation, if present. 27. Lindahl U, Backstrom G, Thunberg L, Leder I G (1980) Evidence for a 3-O sulfated D-glucosamine residue in the antithrombin-binding sequence of heparin. Proc Natl'Acad Sci USA 77: 6551-6555.28. Presta M, Oreste P, Zoppetti G, Belleri M, Tanghetti E, et al. (2005) Antiangiogenic activity of semisynthetic biotechnological heparins: low-molecular-weight-sulfated Escherichia coli K5 polysaccharide derivatives as fibroblast growth factor antagonists. Arterioscler Thromb Vasc Biol 25: 71-76.

One aspect of the present invention is predicated at least in part on findings by the Applicant directed to the synchronization of parasite cultures and the isolation of viable merozoites from Plasmodium, which retain their invasive capacity, at high purity and high yield. This is in contrast to the prior art in which most attempts to purify merozoites that retain their invasive capacity from human malaria parasites have either been unsuccessful, or yielded merozoites with very low invasive capacity, thereby hindering the development of, for example, methods to fix and image merozoites in the process of invasion by standard, fluorescence, or electron. microscopy. The ability to isolate viable merozoites from Plasmodium species such as Plasmodium falciparum, which retain their invasive capacity at high purity and high yield, as described herein allows a significant advancement of knowledge on invasion events and interactions, and facilitates the identification and characterization of inhibitors, such as those described herein, in vaccine and drug development.

Using these methods, Applicant has advanced understanding of merozoite invasive capacity after schizont rupture, the kinetics of invasion, and conditions for invasion. Furthermore, a high-throughput invasion assay has been developed and optimized that can be used to test inhibitory compounds and antibodies.

Previous studies report that merozoites collected from spontaneously ruptured schizonts, usually several hours post-rupture, retain little or no invasive capacity. With reference to the Examples, Applicant has explored whether mature schizonts could be ruptured, and merozoites purified. In one form of the invention highly synchronous mature-stage parasites are isolated, returned to culture and monitored for rupture. Once rupture began to occur, whole parasite preparations are filtered and free merozoites isolated. In this form of the invention, culture of the merozoite preparation with fresh RBCs confirms that a proportion retain invasive capacity, as indicated by the presence of developing intra-erythrocytic parasites.

In another form of the invention, in order to increase the yield of merozoites that retain invasive potential, purified mature-stage parasites are treated with the protease inhibitor trans-Epoxysuccinyl-L-leucylamido(4-guanidino)butane (E64), which prevents merozoite release from schizonts by inhibiting rupture. Applicant found that incubating merozoites with E64 did not affect their invasive capacity. This approach enables a parasite preparation enriched for schizonts to be obtained. After the majority of parasites are fully developed in the presence of E64, parasites are pelleted, resuspended into culture media and merozoites purified by filtration. This aspect of the invention shows that by comparison of merozoites from untreated and E64-treated schizontz, the E64-treated preparations have a surprisingly higher proportion of merozoites that are invasive.

Accordingly, in one aspect, the present invention provides a method of at least partially synchronising a population of two or more Plasmodium-infected cells comprising exposing the two or more Plasmodium-infected cells to a protease inhibitor to inhibit schizont rupture, thereby halting development of the Plasmodium-infected cells at the schizont stage.

In another aspect, the present invention provides a method of isolating Plasmodium merozoite from an at least partially synchronous population produced by the methods described herein comprising rupturing the schizonts to allow merozoite release.

In the context of the present invention, the term “synchronisation” relates to the enrichment of parasites of a particular life cycle stage (e.g. ring stage parasites, trophozoite stage parasites or schizont stage parasites) or enrichment of parasites of a particular age post invasion. Accordingly, the term includes the narrowing of the range of age of a population of parasites, and it would be understood that the term includes different levels of synchronicity. Thus the synchronized populations include, but are not limited to, populations having 50% or more parasites of a particular life cycle stage or age post invasion (e.g. 60%, 65%, 70%, 75%, 80%, 85%, 90%, 91%, 92%, 93%, 94%, 95%, 96%, 97%, 98%, 99%, 99.5% or more).

Similarly, the skilled person understands that the term “isolated” relates to the enrichment of a particular life cycle stage (e.g. merozoites) from other Plasmodium life cycle stages (e.g. schizonts). Accordingly, it would be understood that the term includes different levels of enrichment. Thus the isolated life cycle stage includes, but is not limited to, at least 50% of a particular life cycle stage (e.g. 60%, 65%, 70%, 75%, 80%, 85%, 90%, 91%, 92%, 93%, 94%, 95%, 96%, 97%, 98%, 99%, 99.5% or more).

In one embodiment, the Plasmodium-infected cells are infected with mature stage parasites such as a trophozoite or a schizont.

In one form of the invention experiments are performed with a GFP-expressing D10-PfPHG parasite line to facilitate identification and tracking of merozoites and invasion events. Furthermore, the methods of the present invention have been successfully used to isolate merozoites and obtain invasion of RBCs for the parental D10 and 3D7 lines.

Accordingly, the Plasmodium parasites may be an isolate or strain of Plasmodium, which is a sample of parasites taken from an infected individual on a unique occasion. Typically, an isolate is uncloned, and may therefore contain more than one genetically distinct parasite clone. A Plasmodium falciparum line is a lineage of parasites derived from a single isolate, not necessarily cloned, which have some common phenotype (e.g. drug-resistance, ability to invade enzyme treated red cells etc.). A Plasmodium falciparum clone is the progeny of a single parasite, normally obtained by manipulation or serial dilution of uncloned parasites and then maintained in the laboratory. All the members of a clone have been classically defined as genetically identical, but this is not necessarily the case, since members of the clone may undergo mutations, chromosomal rearrangements, etc, which may survive in in vitro culture conditions. Known strains of Plasmodium falciparum include 3D7, W2MEF, GHANA1, V1_S, RO-33, PREICH, HB3, SANTALUCIA, 7G8, SENEGAL3404, FCC-2, K1, RO-33, D6, DD2, or D10, or any other known or newly isolated strain of Plasmodium, or a genetically modified strain, such as those described herein.

The protease inhibitor can be any inhibitor that inhibits schizont rupture. These include inhibitors of schizont rupture that do not inhibit merozoite invasion (e.g. E-64). The skilled person would appreciate that inhibitors of both schizont rupture and merozoite invasion (e.g. TLCK) would need to be removed from isolated merozoites if the merozoites are to be used in invasion inhibition assays as described herein.

In one embodiment the protease inhibitor is trans-Epoxysuccinyl-L-leucylamido(4-guanidino)butane (E64).

The schizonts are allowed to proceed to rupture as they mature through the Plasmodium life cycle or are ruptured by any means available that results in the release of the merozoites. One form of rupture may be mechanically (e.g., ruptured by filtration).

The released merozoites may be further isolated by filtration. In one embodiment, the schizonts are ruptured and merozoites isolated by filtration, for example using a 1.2 um filter. Forced-rupture of schizonts by filtration facilitates minimizing handling.

Applicant has also demonstrated effective pelleting of merozoites by centrifugation with the viability of pelleted merozoites being maintained. Thus, in one embodiment, merozoites are isolated by centrifugation.

After invading erythrocytes, the parasite converts the heme groups of hemoglobin into an insoluble highly compacted crystal known as “hemozoin”. The conversion is made by the parasite to detoxify the heme. The hemozoin is present in intra-erythrocyte stages of the parasite; the ring, trophozoite, schizont, and gametocyte stages. When the parasite reaches maturity and the erythrocyte burst's during schizont rupture, the hemozoin is released.

Accordingly, in another aspect of the invention, there is provided a population of merozoites that are populations having 50%,60%, 65%, 70%, 75%, 80%, 85%, 90%, 91%, 92%, 93%, 94%, 95%, 96%, 97%, 98%, 99%, 99.5% or more purity.

In yet another aspect, the present invention provides a population of substantially synchronised Plasmodium-infected cells.

In another aspect, the invention provides a population of merozoites from a population of Plasmodium-infected cells wherein the cells are desirably substantially synchronised and the population of merozoites has 50% 60%, 65%, 70%, 75%, 80%, 85%, 90%, 91%, 92%, 93%, 94%, 95%, 96%, 97%, 98%, 99%, 99.5% or more purity. In a further embodiment, the method may further comprise removing haemozoin crystals from the isolated merozoites.

Each heme-group contains a high-spin Fe+3 (S=5/2) stacked in close proximity. The Fe—Fe atomic separation is around 8 angstrom (Andrzej et al., J. Am. Chem. Soc. 128:4534-4535, 2006). The transformation of low-spin (Fe+2) diamagnetic oxyhemoglobin into high-spin (Fe+3) hemozoin and the close proximity of the Fe atoms give rise to the strong paramagnetic properties of the haemozoin.

Accordingly, in one embodinient the haemozoin crystals are removed by exposing the isolated merozoites to a magnetic field.

The magnetic field may be provided by a magnet. The magnets used in the invention are permanent magnets, such as, for example, SmCo and NeFeB magnets. Alternatively, other possible magnets that may be used include, but are not limited to, ceramic magnets (Strontium and Barium Ferrite), flexible magnets neodymium magnets (Nd—Fe—B), samarium magnets and alnico magnets. The permanent magnets may also be substituted with a variety of electromagnetic sources. Electromagnets may be made from materials that include, but are not limited to, copper and superconductive material. In certain embodiments, the magnetic field is applied using an array of magnets. Alternatively, the magnetic field may be applied using a single magnet. Preferably, the magnetic field generated from the use of such magnet ranges from between 0.1-1.0 Tesla and is selected by the skilled artisan based on one or more criteria, such as, for example, the differential magnetic properties of blood components to be separated and viscosity of the blood or blood derived fluid in which those components are located.

This invention provides the ability to generate a population of two or more Plasmodium-infected cells that is highly synchronous, and using treatment with E64 enables the skilled person to obtain a parasite preparation enriched for schizonts and therefore a high yield of purified merozoites.

In another aspect, the present invention provides a method for identifying a compound capable of inhibiting invasion of one or more Plasmodium merozoites into one or more cells, the method comprising exposing the one or more cells to the one or more merozoites in the presence of the candidate compound and determining whether invasion of the one or more merozoites into the one or more cells has occurred.

Applicant has demonstrated that compounds can inhibit invasion in vitro (see Examples). Accordingly, the compound capable of inhibiting invasion may be further capable of inhibiting invasion in vivo. As used herein, the term “invasion-inhibitory” is intended to include the complete prevention of invasion of an invasion-competent erythrocyte. The term is also intended to include the partial prevention of invasion, as measured by for example; the proportion of a population of invasion-competent erythrocytes that are invaded, the number of attempts by which it is necessary for a given parasite to invade an erythrocyte, the time taken for a parasite to invade an erythrocyte, and the number of parasites required to ensure that a single erythrocyte is invaded. The inhibition of invasion may be measured in vivo or in vitro.

For the avoidance of doubt, the term “invasion” is intended to include the entire invasion process such that the complete parasite enters the cytoplasm, and is completely encircled by the cytoplasm. The term also includes components of the entire invasion process such as the binding of the merozoite to the surface of the erythrocyte, the reorientation of the apical end of the parasite to contact the erythrocyte surface, entry of the parasite into a parasitophorous vacuole, release of protein from apical organelles, and the shedding of merozoite surface protein by proteases.

The merozoites may be prepared by a method as described herein.

The one or more cells are erythrocytes (e.g. human erythrocytes). In another embodiment, the one or more cells are reticulocytes.

It was previously thought that merozoite survival post-release was very brief and that merozoites must invade RBCs within seconds or up to 1-2 minutes. Applicants have found that that the half-life of merozoite invasive potential was 5-6 minutes at 37° C., and most invasion events occurred over a period of 10 minutes. This survival period is considerably longer than expected and may be important physiologically. Without wishing to be bound by theory, parasitized-RBCs sequester in vascular beds and are thought to develop through to schizonts while bound to endothelial cells. Presumably, merozoite viability would need to be maintained for several minutes to allow sufficient time for merozoites to enter the circulation and invade RBCs after release by rupture of sequestered schizonts. The duration of survival and kinetics of invasion also have significant implications for the understanding of host-parasite interactions. The persistence of viable extracellular merozoites for several minutes would allow sufficient time for interactions to occur between merozoites and antibodies or circulating immune cells. It may also allow for cellular interactions in the spleen since ˜5% of circulating blood volume passes through the spleen per minute. Therefore, significant numbers of free merozoites may be carried into the spleen, including those that do not successfully invade, which may be important for the development of immune responses.

The relatively short viability of merozoites probably explains why prior attempts to isolate merozoites from naturally-ruptured schizonts have generally been unsuccessful; harvesting merozoites is commonly done several hours post-rupture and involved significant handling and washing steps.

In one embodiment, exposure of the one or more merozoites to the one or more cells is performed at a temperature to maximise the half life of merozoite invasive potential. For example, the method is performed at room temperature (e.g. about 19, 29, 21, 22, 23, 24 or 25° C., preferably around 22° C.).

Of practical importance, the duration of merozoite invasion potential was much greater at room temperature; allowing sufficient time for treatment or manipulation of merozoites before setting up invasion assays, facilitating the ability to investigate and identify potential inhibitors.

In another embodiment, the method is performed at a physiologically relevant temperature (e.g. about 36, 37 or 38° C., preferably 37° C.).

Exposure of the one or more merozoites to the one or more cells is performed for at least the invasive half life of merozoite invasive potential at a particular temperature. In one embodiment, the method is performed for at least 5-6 minutes at about 37° C., in another embodiment the method is performed for at least 10 minutes at about 37° C.

Applicant has demonstrated that the efficiency of merozoite invasion is significantly influenced by the ratio of merozoites to cells. Thus, as the merozoite:RBC ratio decreases the resulting invasion rate (proportion of merozoites that invaded) increases. Maximum invasion rates can be achieved at low merozoite:RBC ratios (i.e. an excess of RBCs). Under conditions of low merozoite:RBC ratios, the parasitemia of post-invasion cultures is low. Higher parasitemias can be achieved with high merozoite:RBC ratios (i.e. an excess of merozoites) with a subsequent decline in the proportion of merozoites that invade.

The ratio of merozoites:RBCs can have a significant effect on invasion rate, being highest with a low ratio. This is reflective of conditions in vivo, in which a low parasitemia is typically observed in human malaria.

Accordingly, in one embodiment, the ratio of merozoites to erythrocytes is >1. In another embodiment, the ratio of merozoites to erythrocytes is <1. In another embodiment, the ratio is 1:1.

Applicant has also demonstrated the effect of hematocrit on invasion rate.

Accordingly, in one embodiment, the erythrocyte concentration is at least 100×10³ erythrocytes/ul. In another embodiment, the erythrocyte concentration is at least 200×10³ erythrocytes/ul or at least 300×10³ erythrocyteul.

Because low merozoite:RBC ratios can result in low parasitemias in the first growth cycle, Applicant has demonstrated that the requirements for invasion efficiency and resulting parasitemia can be controlled. Accordingly, the methods of the present invention may be performed at 2% final hematocrit and high merozoite:RBC ratios, to balance these factors and to obtain high parasitemias for analysis (e.g. FACS analysis), inhibition studies, and imaging. In another aspect, the haematocrit may be 0.05% to 5% or even 0.01 to 25% as determined necessary by the skilled person.

Applicant was able to substantially increase invasion rates by agitating merozoite:RBC suspensions during the period in which invasion occurs.

Accordingly, in one embodiment, the method is performed with agitation to promote invasion of the one or more merozoites into the one or more cells.

Established assays typically measure inhibition of blood-stage growth and cannot specifically measure invasion inhibition by antibodies or compounds. Assays reported to measure inhibition of merozoite invasion still require culture of late-stage parasites and newly-invaded parasites in the presence of inhibitors and therefore could be measuring inhibition of intraerythrocytic growth and/or schizont rupture. The ability to specifically measure invasion-inhibitory activity, separately from total growth inhibition, is important because some antibodies to merozoite antigens are known to inhibit intraerythrocytic development of parasites as well as inhibiting invasion.

Applicant has demonstrated compounds capable of inhibiting invasion include antibodies (e.g. 1F9), small molecules and other compounds (e.g. the sulfated polysaccharide heparin, cytochalasin D, and R1 peptide), and EDTA.

In one embodiment the candidate compound is a sulfated polysaccharide molecule. The candidate compound may be a sulfated polysaccharide molecule according identified or rationally designed according to the methods of the present invention.

Applicant has demonstrated that invasion assays can be performed with invasion being allowed to occur for determined periods of time.

In another embodiment, following exposure of the one or more purified merozoites to the one or more cells in the presence of the candidate compound, a molecule is added to stop any further invasion occurring, followed by determining whether invasion of the one or more merozoites into the one or more cells has occurred.

In one embodiment, the molecule added to stop any further invasion is heparin.

Prior to the present invention, it was not known whether invasion requires, or is enhanced by serum components. The present invention has addressed this. Thus, in one aspect studies of invasion efficiency or inhibition assays can be performed using antibodies or serum components at concentrations that are close to those in vivo, but can also be performed under conditions that require an absence of serum components or protein.

In one embodiment, the method is performed in the absence of serum.

A further advantage is that Applicant's IIAs can be performed using serum or serum components at near-physiological concentrations, which is important for testing human antibodies and understanding their role in vivo.

In another embodiment, the compound capable of inhibiting invasion is identified according to the methods described herein.

In another embodiment, the present invention provides a method for treating and/or preventing an infection with a Plasmodium, the method comprising administering to a subject in need thereof an effective amount of a composition described herein.

In one form of the method of treatment or prevention the subject is a human. The human may be an infant, a child, an adolescent, or an adult. Use of the vaccine may be especially important in women in child-bearing years. Pregnant women, particularly in the second and third trimesters of pregnancy are more likely to develop severe malaria than other adults, often complicated by pulmonary oedema and hypoglycaemia. Maternal mortality is approximately 50%, which is higher than in non-pregnant adults. Fetal death and premature labor are common.

One way of monitoring efficacy for therapeutic treatment involves monitoring Plasmodium falciparum infection after administration of the compositions of the invention.

The uses and methods are for the prevention and/or treatment of a disease caused by Plasmodium (e.g. malaria) and/or its clinical manifestations (e.g. prostration, impaired consciousness, respiratory distress (acidotic breathing), multiple convulsions, circulatory collapse, pulmonary oedema (radiological), abnormal bleeding, jaundice, haemoglobinuria, etc.).

The compositions of the present invention can be evaluated in in vitro and in vivo animal models prior to host, e.g., human, administration. For example, in vitro neutralization and/or invasion inhibition is suitable for testing vaccine compositions (such as immunogenic/immunoprotective compositions) directed toward Plasmodium.

The methods to synchronise populations of plasmodium infected cells and isolate merozoites that retain invasive capacity, and their use in invasion assays, also have applications in proteomics, metabolomics, transcriptional analyses and transfection to obtain higher transfection efficiencies.

In another aspect, the present invention provides a method for identifying an agent capable of treating or preventing Plasmodium infection the method comprising providing a candidate compound, providing a merozoite protein and assessing the ability of the candidate compound to bind to the merozoite protein. Particular examples of merozoite proteins include MSP1, MSP2, Pf12, Pf38, Pf41 and apical membrane antigen 1. Most preferably, the merozoite protein is MSP1.

Experimental work described herein has demonstrated that sulfated polysaccharides such as heparin bind to MSP1 of Plasmodium falciparum, specifically to the fragment (MSP1-42). This binding is inhibited by related compounds that also inhibit erythrocyte invasion, thereby confirming that MSP1 is a relevant therapeutic target for antiplasmodial compounds. Applicant's identification of a target of inhibitory activity may elucidate essential invasion receptor-ligand interactions and vaccine targets. The identification of heparin inhibition of early erythrcicyte invasion events here is further supported with the identification of MSP1 heparin binding ability. MSP1 is a major GPI anchored surface antigen on the merozoite. MSP1 is also though to be essential for invasion, with antibodies against MSP1-19 and MSP1-42 inhibiting invasion in GIA and with this protein being refractory to genetic knockout. The heparin binding ability of MSP1-42 is consistent with heparin inhibition of early initial attachment events of erythrocyte invasion and the inhibition of an essential step of the invasion process.

Data disclosed herein clearly show the specificity of the interaction; only the processed form of MSP1, MSP1-42, and not full length MSP1 is able to bind heparin (although MSP1-42 fragment is contained in full length MSP1), suggesting that a specific conformation is required for binding. The interaction between MSP1-42 was inhibited with compounds which also had growth inhibitory activity, but no by non-inhibitory compounds. Furthermore EBA175, known to bind to erythrocyte sialic-acid motifs is not able to bind even though it binds negatively charged molecules in its native interaction. The binding of native MSP1-42 was further confirmed with binding of recombinant MSP1-42.

### EXAMPLES

### Example 1

### Materials and Methods

### Reagents

Heparin (sodium salt, porcine intestinal mucosa), de-N-sulfated heparin (re-acetyl, sodium salt), de-N de-O-sulfated heparin (re-acetyl, sodium salt), dextran sulfate, fucoidan, chondroitin sulfate C (CSC) and heparin-agarose beads were obtained from Sigma-Aldrich (Australia). Partially de-N-sulfated and re-N-acetylated heparin, de-6-O-sulfated heparin, de-2-O-sulfated heparin, all modified K5 polysaccharide derivatives, including those with N-sulfation (K5-NS), low level of O-sulfation (K5-OS-L), high level of O-sulfation (K5-OS-H), low level of N,O-sulfation (K5-NSOS-L), high level of N,O-sulfation (K5-NSOS-H), and epimerized K5 polysaccharide with low level of N,O-sulfation (EK5-NSOS-L), high level of N,O-sulfation (EK5-NSOS-H), as well as CSB-2,4-OS, and CSB-2,6-OS were obtained from Iduron (Paterson Institute for Cancer Research, University of Manchester, UK). CSE was obtained from Seikakagu (Japan). Biotynlated-heparin (porcine intestinal mucosa) was purchased from Calbiochem (Merck, Australia). Anti-rabbit-HRP and anti-mouse-HRP antibodies were purchased from Chemicon (Australia). Rabbit anti-bovine albumin (BSA) was obtained for Sigma-Aldrich (Australia). MSP1-19 antibody and EBA175 antibodies were polyclonal antibodies raised in rabbits against the respective proteins. Recombinant MSP1-42, MSP1-19, AMA1 were expressed in E. coli and purified and re-folded using established methods. Lactoferrin and heparin-BSA were purchased from commercial sources. Enzymes neuraminidase (Vibrio cholerae) was purchased from Calbiochem (Merck, Australia), chymotrypsin (Bovine pancreas) and trypsin (Bovine Pancreas) were from Worthington Biochemical Corporation (NJ, USA).

### Preparation and Characterization of Heparin and Chondroitin Sulfate C Oligosaccharide Fragments.

Heparin and chondroitin sulfate (CS) C oligosaccharide fragments where prepared and characterized as described previously with 30% completion for heparin [29] and 60% completion for CSC [30]. Briefly, 200 mg of heparin was incubated with heparinase I (100 units) in 5 mM sodium phosphate buffer (pH 7.1, 6 ml) containing 0.2 M NaCl and BSA (1 mg) and CSC (200mg) was digested with 0.5 U of chondroitinase ABC in 50 mM, pH 7 sodium phosphate buffer with 0.2 M NaCl. The reaction was carried out at 30 ° C. and stopped when digestion was 30% complete for heparin and 60% complete for CSC, as monitored by UV absorbance at 232 nm. The digests were de-salted on a short Sephadex G10 column. Fractionation of heparin oligosaccharides was carried out on a Bio-Gel P-6 column (1.6×90 cm) with elution by 0.2 M NH4Cl (pH 3.5) while the CSC digest was fractionated on a Bio-Gel P-4 column (1.6×90 cm) with elution by 0.2 M ammonium acetate. Eluate was monitored on-line by refractive index and UV at 232 nm. Oligosaccharide fractions were collected, desalted and freeze-dried before quantitation by carbazole assay for hexuronic acid content [31]. The oligosaccharide fractions were re-suspended in human tonicity phosphate-buffered saline (HT-PBS) and filter sterilized before inhibition assay. 29. Chai W, Luo J, Lim C K, Lawson A M (1998) Characterization of heparin oligosaccharide mixtures as ammonium salts using electrospray mass spectrometry. Anal Chem 70: 2060-2066.30. Beeson J G, Chai W, Rogerson S J, Lawson A M, Brown G V (1998) Inhibition of binding of malaria-infected erythrocytes by a tetradecasaccharide fraction from chondroitin sulfate A. Infect Immun 66: 3397-3402.31. Bitter T, Muir H M (1962) A modified uronic acid carbazole reaction. Anal Biochem 4: 330-334.

### Parasite Culture

P. falciparum was cultured as described [32]. Parasites were maintained in culture media of RPMI-HEPES supplemented with 5% (vol/vol) heat inactivated (56° C. 1 hr) human serum (Australian Red Cross Blood Bank),. 5% albumax (Gibco), 10mM L-glutamine (Gibco) and 25 mM Na2HCO3. Parasites were grown at 37° C., 2% hematocrit, O+ human erythrocytes (Australian Red Cross Blood Bank), 1% O2 4% CO2 95% N2 atmosphere. Sorbitol (Sigma-Aldrich) was used to synchronize cultures [33]. 32. Persson K E, Lee C T, Marsh K, Beeson J G (2006) Development and optimization of high-throughput methods to measure Plasmodium falciparum-specific growth inhibitory antibodies. J Clin Microbiol 44: 1665-1673.33. Lambros C, Vanderberg J P (1979) Synchronization of Plasmodium falciparum erythrocytic stages in culture. J Parasitol 65: 418-420.

### Growth Inhibition Assays

Heparin and K5 polysaccharides (Table 1) were tested for inhibitory activity against P. falciparum parasites using high throughput growth inhibitory assays (GlAs) [32]. Duplicate 25 μl suspensions of synchronized parasites, 2% parasitemia (32 hr trophozoite), 1% hematocrit with normal or enzymatically treated erythrocyctes, were incubated with inhibitory compounds in 96 well sterile U-bottom plates (Falcon). For humidification, outside wells were filled with sterile human tonicity phosphate-buffered saline (HT-PBS). Plates were incubated as for parasite culture for 44 hrs and analyzed. Parasites were stained with 10 μg/mL ethidium bromide (EtBr) (BioRad), HT-PBS, 1 hr in darkness. Cells were washed and re-suspended in 200 μL HT-PBS. Parasitemia was measured using FACSCalibur flow-cytometry. Data were analyzed using FlowJo (Tree Star), gating on intact erythrocytes and determining parasitemia by EtBr staining. Inhibitory effects of compounds were expressed as percent growth of uninhibited controls (HT-PBS) for each experiment.

### Enzyme Treatment of Erythrocytes

Erythrocytes were treated with neuraminidase, chymotrypsin and trypsin as described previously [34]. Erythrocytes were washed with RPMI-HEPES and incubated with neuraminidase (67 μM, 15 minutes), chymotrypsin (1 mg/mL, 45 minutes) or trypsin (100 μg/mL, minutes) at 37° C. Neuraminidase treated bells were washed three times with RPMI-HEPES and chymotrypsin and trypsin treated cells were washed once with culture media and twice with RPMI-HEPES and then used in GIAs. 34. Persson K E, McCallum F J, Reiling L, Lister N A, Stubbs J, et al. (2008) Variation in use of erythrocyte invasion pathways by Plasmodium falciparum mediates evasion of human inhibitory antibodies. J Clin Invest 118: 342-351.

### Time Course of Schizont Rupture and Merozoite Invasion/Ring Formation

50 μL of synchronous 3D7 parasites at high parasitemia (5-10%), at approximately 32 hrs trophozoites were incubated as for GIAs with or without inhibitors (100 μg/mL). From approximately 44-48 hr schizonts parasite samples were collected, every 3 hrs for 15 hrs. Cells were stained and analysed as for GIAs to evaluate parasitemia and parasite stage.

### Live Filming of Parasite Invasion

Parasites cultures of 5-10% late stage schizonts were filmed with video microscopy in normal culture media, 37° C. 6% CO2 with humidification with or without 100 μg/mL of inhibitory compounds added to cultures as described previously [35]. 35. Gilson P R, Crabb B S (2009) Morphology and kinetics of the three distinct phases of red blood cell invasion by Plasmodium falciparum merozoites. Int J Parasitol 39: 91-96.

### Binding of Native Proteins to Heparin-Agarose Beads

Synchronized schizont stage (40-48 hr, 5-8% parasitemia) cultures were used for protein extraction. Uninfected erythrocytes were lysed with 0.15% saponin (Kodak) on ice for ten minutes. Remaining pellets were washed three times with cold HT-PBS. Protein from schizonts was extracted with 5 times volume of 1% TritonX100 (TX100) (Sigma-Aldrich, Australia), HT-PBS with protease inhibitors (Roche). Insoluble components were pelleted by centrifugation and supernatants collected. Schizont TX100 extracted proteins were bound to heparin-agarose beads (Sigma-Aldrich, Australia) as previously described [36]. Heparin-agarose beads were washed once with 1% casein in PBS, once in PBS, and then blocked with 1% casein in PBS overnight at 4° C. TX100 merozoite protein extracts where incubated with beads containing 0.1% casein and 200 μg/mL of test inhibitor, or PBS as control, overnight at 4° C.; 50 μL of packed beads and 100 μL protein and inhibitor were used or each test sample. Inhibitors used were heparin and CSC. Unbound proteins in the supernatant were collected through Micro Bio-Spin Chromatography Columns (Bio-Rad). After incubation, beads were washed five times with PBS containing 0.1% casein and 1% Triton X-100. Bound proteins were eluted from beads with 50 μL of warmed reducing sample buffer. Bound and unbound proteins were separated by SDS-PAGE under reducing conditions and blotted onto membranes for probing with antibody detection with anti-MSP1 or anti-EBA175. 36. Baum J, Chen L, Healer J, Lopaticki S, Boyle M, et al. (2009) Reticulocyte-binding protein homologue 5—an essential adhesin involved in invasion of human erythrocytes by Plasmodium falciparum: Int J Parasitol 39: 371-380.

### Heparin-BSA Binding Assay

Recombinant merozoite antigens were coated (1 μg/mL) onto 96 well plates (Nunc Maxisorb) in HT-PBS overnight at 4° C. Plates were washed and blocked with 1% casein, 1 hr, room temperature. Plates were incubated with heparin-BSA conjugate with soluble inhibitors and detected with anti-BSA (rabbit) (Sigma-Aldrich) antibodies and anti-rabbit -HRP. Detection was performed with 2,2′-azino-bis(3-ethylbenzthiazoline-6-sulphonic acid) (Sigma-Aldrich) monitored for colour change and measured for absorbance at 405 nm. All incubations were performed in HT-PBS 0.1% casein 0:05% Tween20, 1 hr at room temperature. Plates were washed 3 times HT-PBS 0.05% Tween20 between incubations.

### Modeling of Glycosaminoglycan 3D Structures

Representative 3D structures of heparin and heparin-like compounds were developed using SWEET-II http://www.glycosciences.de/modeling/sweet2/doc/index.php [37] based on 3 disaccharide units of most abundant uronic acid/amino sugar/sulfation residues in compounds. For graphical display the plug-in Chime was used http://www.mdl.com/products/framework/chime/index.jsp. Hydrophobicity models were generated as relative molecular lipophobicity potentials as defined by RASMOL. 37. Bohne A, Lang E, von der Lieth C W (1999) SWEET—WWW-based rapid 3D construction of oligo- and polysaccharides. Bioinformatics 15: 767-768.

### Example 2

### Heparin Inhibits Initial Contact and Reorientation of Merozoites During Erythrocyte Invasion

To define the mechanism of action of heparin and heparin-like molecules Applicant performed time-course studies of schizont rupture, merozoite invasion and intra-erythrocytic development of P. falciparum in the presence of heparin. Parasite stages were evaluated using flow-cytometry, differentiating between late stage trophozoites and schizont versus ring stage parasites with ethidium bromide staining. In the presence of heparin (100 μg/mL), there was evidence of some delay, but not complete inhibition, of schizont rupture compared to control (FIG. 7A, B). In contrast, rates of merozoite invasion and ring formation were very low, suggesting heparin acts primarily by blocking invasion. No inhibition of infra-erythrocytic growth was observed (data not shown).

A mechanism of action of heparin was next determined using live time-lapse microscopy. In control parasite cultures, schizont rupture and step-wise invasion of erythrocytes by merozoites was readily observed (FIG. 7C). During merozoite invasion of erythrocytes, Applicant observed initial contact with erythrocyte surface resulting in brief deformation of erythrocytes, merozoite reorientation, mechanical invasion resulting in erythrocyte deformation, and erythrocyte re-shaping upon complete invasion, as reported previously [35,38,39]. In the presence of heparin (100 μg/mL) schizont rupture occurred and merozoites appeared to disperse normally further establishing that heparin does not act by inhibiting schizont rupture (FIG. 7D). Initial contact of merozoites with the erythrocyte surface was also observed; however, contact between erythrocytes and merozoites was not sustained, and merozoites dissociated after various lengths of time (FIG. 7D). No successful invasion events were observed; additionally, reorientation of merozoites and deformation of erythrocytes were not clearly seen, although the observation of merozoite reorientation is limited due to microscope resolution. The accumulated filming time of control parasites was 5442 seconds covering 13 separate schizont rupture events, with 21 confirmed invasions. Accumulated filming time of cultures with heparin was 4529 seconds over 15 separate events, with no successful invasion observed. These studies clearly establish that heparin acts by inhibition of invasion and blocking initial events of erythrocyte invasion. 38. Dvorak J A, Miller L H, Whitehouse W C, Shiroishi T (1975) Invasion of erythrocytes by malaria merozoites. Science 187: 748-750.39. Glushakova S, Yin D, Li T, Zimmerberg J (2005) Membrane transformation during malaria parasite release from human red blood cells. Curr Biol 15: 1645-1650.

### Example 3

### Heparin Inhibits Essential Erythrocyte Invasion Events

Heparin inhibited all parasite lines tested, including genetically different lines 3D7, W2mef, D10, E8B, XIE, HCS3) (data not shown). Parasite lines W2mef and 3D7 were used as representative of sialic-acid (SA)-dependent and SA-independent invasion phenotypes, respectively; both were inhibited at equal levels (FIG. 8A). To specifically examine heparin activity against different invasion phenotypes or pathways, Applicant measured inhibition of merozoite invasion by heparin using erythrocytes treated with enzymes to selectively remove subsets of erythrocyte invasion receptors. With 3D7 parasites the activity of heparin was equivalent when tested for inhibition of merozoite invasion into neuraminidase, trypsin, chymotrypsin, and control-treated erythrocytes (FIG. 8B), suggesting heparin inhibits essential invasion events. Furthermore, chymotrypsin treatment of erythrocytes had no effect on the inhibitory activity of heparin against W2mef parasites (data not shown). Of note, heparin effectively inhibited invasion of merozoites into neuraminidase-treated erythrocytes, indicating that heparin does not act by interfering with the binding of parasite ligands to SA on the erythrocyte surface. Applicant also found that heparin was effective at inhibiting parasites with deletions of the SA-binding ligands EBA175, EBA140, or EBA181 (data not shown).

In the presence of heparin 100 μg/mL, a very low level of parasite growth was observed over one cycle of replication. It was possible that this represented parasites that were resistant to heparin inhibition. In order to select for possible heparin-resistant parasites, Applicant re-cultured parasites present after 48 hour incubation in heparin until a high parasitemia was obtained; and these parasite were then again treated with heparin for 48 hours and surviving parasites were re-cultured. When the parent and heparin-selected (1 and 2 times) cultures were tested in growth-inhibition assays, the sensitivity to heparin was found to be the same; there was no evidence that Applicant were able to select for increased resistance to heparin (FIG. 8C). Additionally, attempts to select for increased resistance by long-term culture with low concentrations of heparin were not successful (data not shown). This further suggests that heparin inhibits essential interactions during invasion.

### Example 4

### IC₅₀ of Heparin is Reduced with Complementary Inhibitory Compounds, Pyrimethamine and AMA1 Binding Peptide

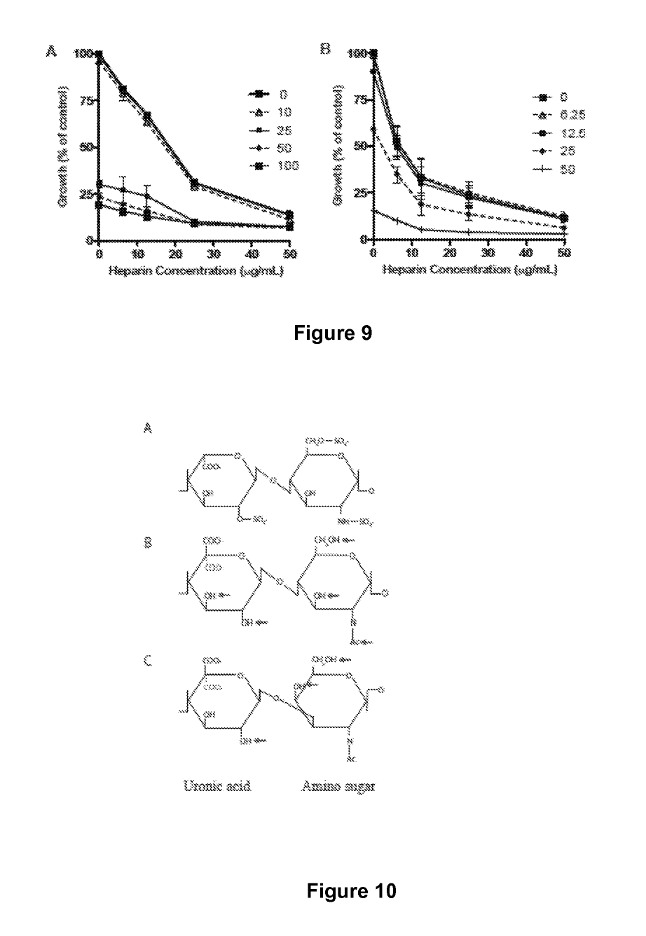

Heparin was tested in combination with pryimethamine or AMA1-binding peptide for inhibition of P. falciparum to further understand mechanism of inhibition and to investigate the potential value of drug combinations. Pyrimethamine is commonly used in combination with sulfadoxine against malaria infection, and is active against intra-erythrocytic growth. In growth inhibitory assays, Applicant found that heparin and pyrimethamine acted in an additive manner, with combinations reducing the IC₅₀ concentration of heparin (FIG. 9A). AMA1-binding peptide inhibits the function of AMA1 and blocks merozoite reorientation and invasion [40]. A combination of heparin with AMA1-binding peptide was highly effective and significantly reduced the concentration of heparin needed to gain 50% inhibition (i.e. IC₅₀ heparin alone 19 μg/mL, AMA1-binding peptide ˜30 μg/mL, when AMA1-binding peptide is 6.25 μg/mL, IC₅₀ of heparin ˜3 μg/mL) (FIG. 9B). Furthermore, the additive effect of heparin and AMA1 binding peptide suggests that heparin is acting through a different target from AMA1. These data show that heparin has the potential to be used in combination with complementary drugs to reducing active IC₅₀ further highlighting potential therapeutic value. 40. Harris K S, Casey J L, Coley A M, Masciantonio R, Sabo J K, et al. (2005) Binding hot spot for invasion inhibitory molecules on Plasmodium falciparum apical membrane antigen 1. Infect Immun 73: 6981-6989.

### Example 5

### The level and Pattern of Sulfation are Linked to the Inhibitory Activity of Heparin and Heparin-Like Molecules

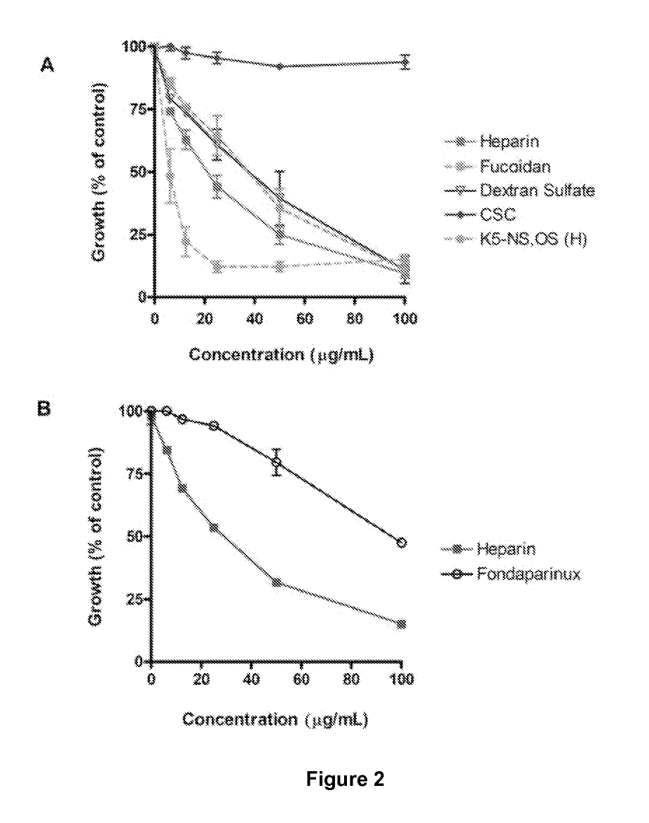

To investigate the structural basis for the inhibitory activity of heparin against P. falciparum blood stage growth, a range of heparin and related sulfated polysaccharides was tested in growth inhibitory assays (GIAs) against the 3D7 parasite line. This included variously modified heparins and K5 polysaccharides (FIG. 4). The sulfation levels and sulfation patterns of these compounds (supplied by manufacturers or as described previously [13]) is shown in Table 1. Dextran sulfate, fucoidan, and CSC were also tested. In agreement with previous reports, heparin, dextran sulfate and fucoidan inhibited P. falciparum blood stage growth with IC₅₀ 19, 37 and 38 μg/mL, respectively, over one cycle of parasite growth (FIG. 2). The structurally related glycosaminoglycan CSC was non-inhibitory and was included in all subsequent assays as a negative control.

Completely desulfated and de-N-sulfated heparin had no activity against P. falciparum in GIAs (FIG. 4A), indicating that the inhibitory activity requires the presence of sulfate groups. Comparison of fully de-N-sulfated heparin and partially de-N-sulfated heparin further indicated the involvement of sulfation level in inhibition, with higher activity observed with increasing sulfate level (FIG. 4A). De-6-O-sulfated heparin and de-2-O-sulfated heparin also had reduced activity, suggesting the specific importance of O-sulfation; de-2-O-sulfated heparin had greater inhibitory activity than de-6-O-sulfated heparin (IC₅₀ 123 and 373 μg/mL, respectively). This suggests that spatial positioning of sulfate groups is linked to activity, as these compounds are otherwise similar.

The roles of N- versus O-sulfation, and the overall level of sulfation, was further investigated using K5 polysaccharides with varying levels and patterns of sulfation. K5 polysaccharides with N-sulfation only (i.e. no O-sulfation; d.o.s=1), had no growth inhibitory activity (FIG. 4B). In contrast, highly O-sulfated K5 (K5-OS-H; d.o.s=3) had significant activity (IC₅₀ 20 μg/mL) despite lacking N-sulfation. This indicates that N-sulfation may not be essential for activity. K5 with lower levels of O-sulfation (K5-OS-L; d.o.s=1.4) exhibited lower inhibitory activity, further emphasizing the role of overall level of sulfation. K5 polysaccharides with both N- and O-sulfation (K5-NSOS-L), and K5-NSOS-H) had substantial activity (IC₅₀ 55.7 and 7.4 μg/mL, respectively) (Table 1; FIG. 4C); the greater level of sulfation of K5-NSOS-H compared to K5-NSOS-L was associated with much greater activity. K5-NSOS-H was the most inhibitory of all the compounds tested. Comparing the inhibitory activity of K5-NSOS-H to the K5-OS-H suggests that N-sulfation together with a high level of O-sulfation leads to substantially greater. activity. Taken together with data on the differing activity of modified, heparin compounds, these results indicate the significance of the overall level of sulfation for inhibitory activity against P. falciparum, and suggest that the pattern of sulfation is a parameter linked to activity. Antiplasmodial activity is increased in polysaccharides having an overall degree of sulfation of greater than or equal to 1.5 (eg. d.o.s. ≧1.5, CSE, K5-NSOS-L, K5-NSOS-H, K5-OS-H) for activity, and that these sulfate groups may need to be spatially positioned on the same monosaccharide of the disaccharide (HexA or Glc/GalNAc).

### Example 6

### Role of Chain Length and Uronic Acid or Hexosamine Type in Inhibitory Activity

K5 polysaccharide contains GlcA only, whereas heparin contains mainly IdoA; the inhibitory activity of sulfated K5 polysaccharides suggested that IdoA may not be essential for activity. To investigate the influence of hexuronic acid type on inhibitory activity Applicant tested epimerized K5 (EK5) polysaccharide derivatives. EK5-NSOS-L and EK-NSOS-H were prepared from K5-NSOS-L and K5-NSOS-H, respectively, and contain an estimated 50:50 of IdoA to GlcA ratio (Table 1). Both epimerized K5 derivatives had significantly less inhibitory activity compared to the parent GlcA-containing polysaccharides (FIG. 5C, Table 1). This suggests that IdoA may not be required for optimal activity, and there may be a preference for GlcA. Importantly, K5 polysaccharides with GlcA have little anticoagulation activity [13 ] and may be more suitable for development as potential antimalarials.

Chondroitin sulfate compounds contain galactosamine rather than glucosamine as the hexosamine residue in the polysaccharide chain (Table 1). To investigate the structural role of hexosamine residues in antiplasmodial activity a number of chondroitin sulfate compounds were tested in GIAs. Test compounds were CSC, CSD, CSE, CSB-2,4-OS and CSB-2,6-OOS (Table 1C and Table 2B). CSE was the only CS compound with substantial GIA activity with IC₅₀ of 26.7 μg/mL. The activity of CSE shows that GlcNAc is not an absolute requirement of antiplasmodial activity and further strengthens the Applicant's demonstration of the structure/function relationship of di-sulfation of a single monosaccharide unit being required for increased activity.

To investigate the role of chain length in activity, heparin and CSC oligosaccharides (2 to 16 monosaccharide units in length) were tested. A minimum of 6 monosaccharide units of heparin was needed for inhibitory activity (FIG. 5D), and 8 mers or higher oligosaccharides had greater activity (IC₅₀ 79, 67, and 67 μg/mL, for 6 mer, 8 mer, and 10 mer, respectively (data not shown)). CSC oligosaccharides had little or no inhibitory activity compared to heparin oligosaccharides of the same length.

### Example 7

### Binding of Native MSP1-42 to Heparin and Inhibition by Invasion-Inhibitory Compounds

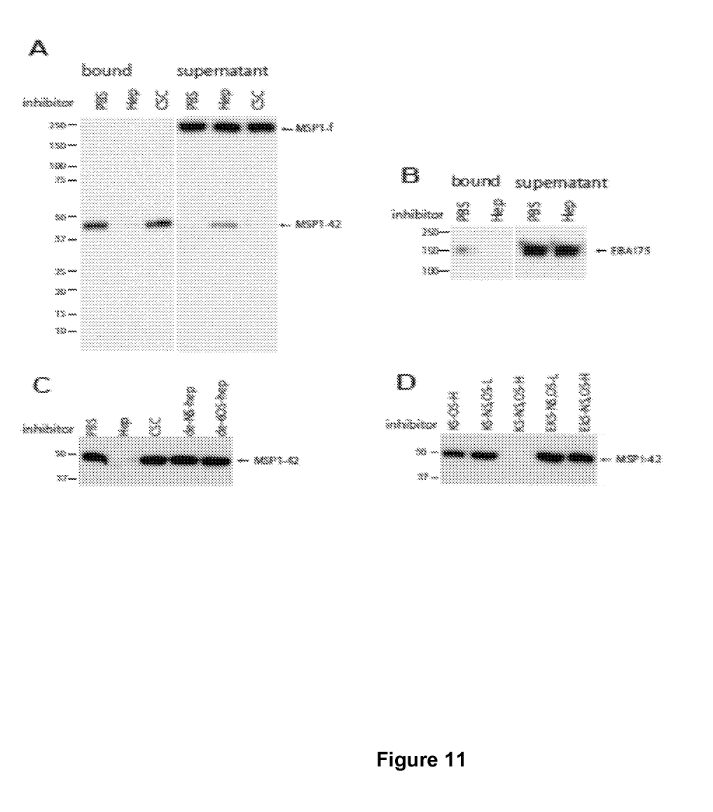

Consistent with the timing of action of heparin in inhibiting initial invasion events, Applicant found that MSP1 had heparin-binding activity. MSP1 is thought to be important in initial attachment of merozoites to the erythrocyte surface and appears to be involved in erythrocyte invasion. MSP1 exists as a high molecular mass protein (Mr ˜180 kDa) and is proteolytically processed into 83 kDa, 30 kDa, 38 kDa and a C-terminal 42kDa (MSP1-42) fragment, held together on the surface of the merozoite by non-covalent bonds. During invasion, further processing occurs of MSP1-42 to remove all but a short C-terminal fragment (known as MSP1-19) on the merozoite surface, which is carried inside the erythrocyte. Processing is thought to be required for MSP1 function and erythrocyte invasion, and data suggests that MSP1-42 and MSP1-19 play important roles in invasion. Applicant tested native MSP1, extracted from P. falciparum merozoites, for binding to immobilized heparin and evaluated the specificity of binding using defined inhibitors. Extracted proteins were incubated with heparin-agarose beads and bound proteins were eluted from beads and then identified by Western blotting. Using this approach Applicant consistently identified in repeat experiments a MSP1 positive band in the bead-eluted fraction that had a Mr of ˜40 kDa; this represents MSP1-42 (FIG. 11A, PBS control). The same band was readily detected in protein extracts prior to incubation with beads, but was greatly depleted from the supernatant after incubation with heparin-agarose beads. In contrast, there was little or no binding of full-length MSP1 in the same preparations. Both forms of MSP1 were detected in schizont protein extracts, but there was little or no detectable protein in fractions eluted from beads, and little evidence of depletion of these proteins from the supernatant following incubation with beads (FIG. 11A). The binding of MSP1-42 to heparin-agarose beads was effectively inhibited by soluble heparin, but not CSC; showing that the binding was unlikely due to non-specific binding to beads (FIG. 11A, heparin and CSC inhibitors). Another merozoite protein, EBA175, did not appear to bind to heparin-agarose beads and was consistently observed predominately in the supernatant after incubation of the protein extract with beads (FIG. 11B). With extended exposures of blots some EBA175 was observed in the bound fraction, but this was far less than in supernatant, suggesting there was little or no specific binding of this protein. EBA175 binds to SAs, which are also negatively charged like heparin. Its lack of binding to heparin supports the specificity of the heparin-binding assay.

Binding of MSP1-42 to heparin-agarose beads was effectively inhibited by soluble heparin, but not by de-N-sulfated heparin, de-6-O-sulfated heparin or CSC (FIG. 11C), establishing the specificity of the interaction and demonstrating a dependence on sulfation for binding. Binding of MSP1-42 was also inhibited by soluble K5-NSOS-H and to a lesser degree by K5-OS-H. There was very little or no inhibition of binding by K5-NSOS-L, EK5-NSOS-L, or EK5-NS,OS-H (FIG. 11D). The binding-inhibition activity of the polysaccharides closely reflected their activity in cellular inhibition assays further suggesting that binding to MSP1-42 is likely to be, at least in part, mediating the growth-inhibitory effect of heparin and heparin-like molecules.

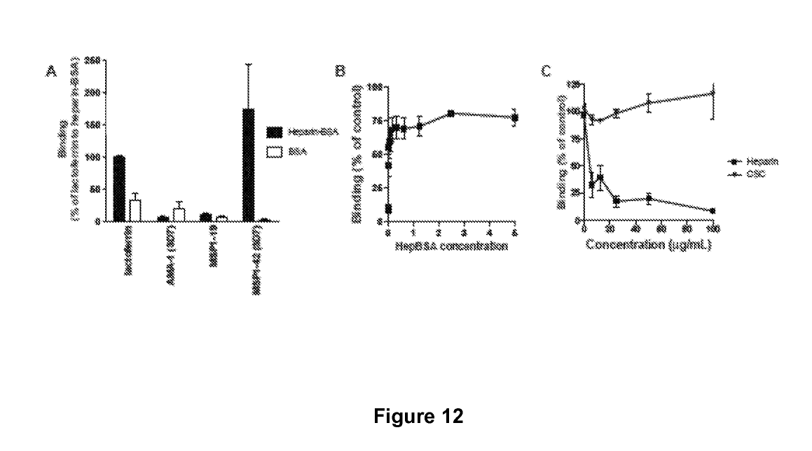

To further examine the heparin-binding activity of MSP1, Applicant tested recombinant MSP1-42 and MSP1-19, as well as AMA1, for binding of heparin conjugated to BSA (heparin-BSA), using BSA alone as a negative control and lactoferrin, a known heparin-binding protein, as a positive control. This revealed that recombinant MSP1-42, but not MSP1-19 or AMA1 had significant heparin-binding activity (FIG. 12A). The binding of lactoferrin to heparin-BSA was dose-dependent and saturated at concentrations of heparin-BSA above 2.5 μg/mL, and binding was inhibited by heparin, but not CSC (data not shown); this validated the use of this assay in studying heparin-binding interactions. Binding of heparin-BSA to MSP1-42 was similarly dose-dependent and saturated with concentrations approximately 1 μg/mL and higher (FIG. 12B). MSP1-42 binding was inhibited in a concentration-dependent manner by heparin, but not by CSC (FIG. 12C). Furthermore, there was inhibition by de-sulfated or de-6-O-sulfated heparin (data not shown).

### Example 8

### Merozoite Purification and Invasion Inhibition Assays—Materials and Methods

### Parasite Culture and Synchronization

P. falciparum isolates were cultured as described supra, in RPMI-HEPES culture medium containing 10% pooled human serum. The GFP-labeled parasite line D1OPfPHG [Wilson D, Crabb B, & Beeson J (2010) Development of fluorescent Plasmodium falciparum for in vitro growth inhibitor assays. Malaria Journal In press] was used in most experiments due to its 48 hour life-cycle, which facilitated obtaining synchronous cultures, and expression of cytosolic GFP allowing for enhanced detection by flow cytometry and fluorescence microscopy. Parasites were synchronized using sorbitol-treatment [Lambros C & Vanderberg J P (1979) Synchronization of Plasmodium falciparum erythrocytic stages in culture. J Parasitol 65(3):418-420] and by using the invasion inhibitory properties of heparin described supra. Parasites were cultured in the presence of 30 IU, of medical grade heparin (Porcine mucous, Pfizer) (approximately 230 ug/ml) until the majority of parasites were at the schizont stage. Heparin was then removed from cultures for 4-6 hours allowing schizont rupture and merozoite invasion to occur. After the invasion period, heparin was added to cultures resulting in the blocking of any further invasion events.

### Merozoite Purification

P. falciparum isolates were cultured as described supra, in RPMI-HEPES culture medium containing 10% pooled human serum. The GFP-labeled parasite line D10-PfPHG was used in most experiments due to its 48 hour life-cycle, which facilitated obtaining synchronous, cultures and reliable prediction of the timing of schizont rupture for harvesting merozoites. Furthermore, this parasite line expresses cytosolic GFP allowing for enhanced detection of merozoites and infected RBCs by flow cytometry and fluorescence microscopy.

Parasites were initially synchronized using sorbitol-treatment, as described. Following sorbitol-synchronization cultures were further synchronised using the invasion inhibitory properties of heparin, to inhibit the formation of ring-stage parasites while allowing the maturation of parasites from trophozoite to schizont stage. To do this, parasites were cultured in the presence of 30 IU (approximately 230 ug/ml) of medical grade heparin (Porcine mucous, Pfizer) until the majority of parasites were at the schizont stage; invasion of erythrocytes by merozoites released from any ruptUring schizonts was inhibited by heparin. Heparin was then removed from cultures (by centrifugation of the culture suspension and resuspension of cells in new culture media without heparin) and incubated for 4-6 hours allowing merozoite invasion to occur. After the invasion period, heparin was again added to cultures to block any further invasion events.

Parasites were then incubated until most parasites had developed to late-stage pigmented trophozoites and schizonts (typically 40-42 hours). Early to mid schizont stage parasites (40-44 hours post invasion) were separated from uninfected RBC and early-stage infected RBCs (resulting in >95% purity) with a MAC magnet separation column (Macs Miltenyi Biotec). Purified schizonts were then incubated with 10 uM of E64 (Sigma) for 7-8 hours; Applicant typically used approximately 30 ml of culture media for every 100 ml of starting culture (3% hematocrit, 3% parasitemia). After approximately 6 hours of incubation with E64, treated schizonts were pelleted by centrifugation at 1900 g for 5 minutes. Supernatant was removed and schizonts were resuspended in fresh culture media. Resuspended E64 treated parasites were filtered through a 1.2 um Acrodisc 32 mm syringe filter (Pall Corporation). The filtrate contained free merozoites and hemozoin crystals, and was used in invasion assays, immunofluorescence microscopy, and electron microscopy as described in the manuscript.

### Merozoite Invasion Assays

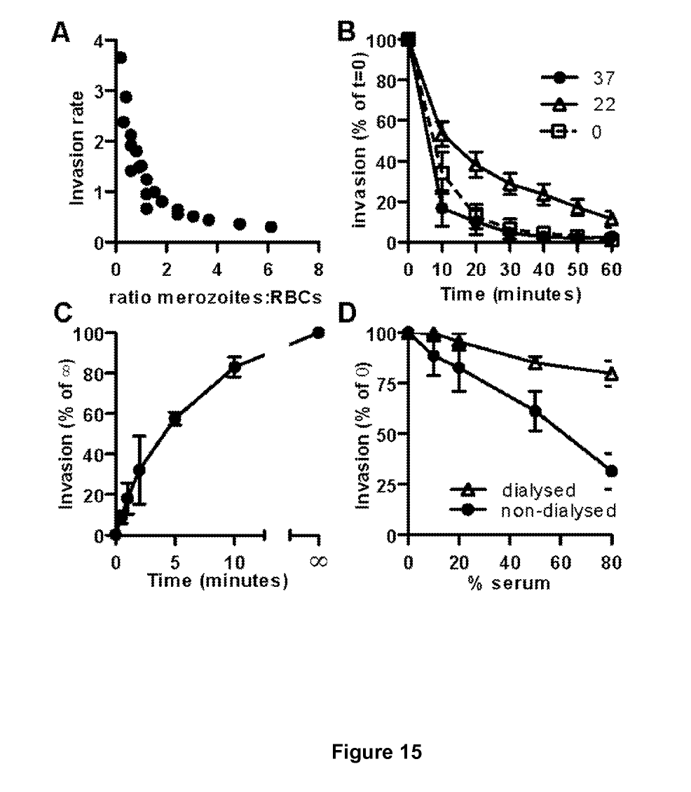

Purified merozoites were mixed with uninfected RBCs and cultured using standard conditions in 96 well U-bottom plates at a final volume of 50 μl or 100 μl per well. Specific assay conditions were varied for different test conditions, as described in the Results section of the manuscript. Assays were typically performed under static conditions. Some assays were also performed with agitation of merozoite:RBC suspensions. Agitation of cell suspensions was performed at 400 rpm on a plate-shaker for 10 mins after mixing merozoites and RBCs as most invasion events were found to occur in the first 10 minutes (see e.g. FIG. 15).

The concentration of merozoites and RBCs used in each individual assay was determined using CountBright Absolute Counting Beads as per manufacturer's protocol (Invitrogen). An identical volume of CountBright beads was added to ethidium bromide stained (5 ug/ml, Biorad) merozoites and uninfected RBC preparations diluted in PBS. Samples were mixed thoroughly and counted using a FACSCalibur flow cytometer (Becton Dickinson) with a minimum of 2000 bead counts collected. Analysis was performed using FlowJo software (Treestar Inc) with beads gated in FL1/FL2, merozoites gated by size in SSC/FSC followed by fluorescence in FL1(GFP)/FL2(EtBr) and uninfected RBCs gated in FL2/FSC. The concentration of merozoites and RBCs was determined by estimating the volume of sample run through the flow cytometer, which was calculated from the number of beads counted, since these were at a known concentration.

To calculate invasion rate of merozoites (defined as the percentage of merozoites that successfully invaded), suspensions of purified merozoites and uninfected RBCs were cultured for 30-40 hours post-mixing; analysis at this time allowed clear separation of late stage parasites from merozoites that had not invaded and cellular debris. Cells were stained with ethidium bromide (10 ug/ml) and analysed by flow cytometry.

The invasion rate of merozoites in filtrate was then calculated as follows:

Invasion rate (% of merozoites successfully invading)=% RBCs invaded (parasitemia)×[(RBCs/ul)/(merozoites/ul)].

To determine the maximum invasion rate of purified merozoites, RBCs and merozoites were mixed at a range of different merozoite:RBC ratios in culture media warmed to 37° C., cultured for 40 hours in 96 well plates under standard conditions, and then parasitemias were measured using flow cytometry, as described above. Maximum invasion rate was achieved at low merozoite:RBC ratio (excess RBCs, see Results); however, it should be noted that this resulted in low parasitemias (% of invade RBCs) due to the large excess of RBCs. Therefore, for our studies we generally balanced requirements for invasion efficiency and resulting parasitemia and performed assays at 0.5% to 1% final hematocrit and high merozoite:RBC ratios to obtain high parasitemias for FACS analysis, inhibition studies, and imaging. To measure the invasive half-life of merozoites, E64-treated schizonts were resuspended in culture medium (room temperature) and filtered to isolate merozoites. Aliquots of the merozoite filtrate were incubated on ice, at room temperature (22 to 23° C.) or at 37 uC. At ten minute time points, merozoites from each temperature treatment were mixed with pre-aliquoted RBCs in a 96 well plate (40 ul of merozoite filtrate was added to 10 ul of uninfected RBC 5% haematocrit, resulting in a 1% final haematocrit). The plate was gassed and incubated at 37° C. between time points. Parasite cultures were then incubated for approximately 40 hours and analysed to calculate parasitemias, as described above. The invasive half-life of merozoites was found. to be longest when incubated at room temperature. Therefore in subsequent assays, purification and handling of merozoites was performed at room temperature.

To define the kinetics of invasion, the invasion inhibitor heparin was used to block merozoite invasion. E64-treated schizonts were resuspended and filtered in culture medium and merozoites were immediately mixed with pre-aliquoted RBCs in 96 well plates. The plate was warmed on a 37° C. heat block to ensure invasion occurred. Heparin was added (200 μg/ml final concentration) at. regular time points to block invasion. After the final time point, cells were incubated and resulting parasitemias were analysed 40 hours later.

To investigate the effect of human serum on merozoite invasion, E64 treated schizonts were resuspended in culture media without human serum and filtered to purify merozoites. Small volumes (10 ul) of filtrate containing merozoites was added to uninfected RBCs (10 ul at 10% hematocrit) made up in culture media without serum. Human serum (serum was pooled from multiple malaria non-exposed Melbourne donors) and/or culture media with no serum was added to obtain final human serum concentrations of 0, 10, 20, 50 and 80% in a final volume of 100 ul. Serum was used with or without dialysis against RPMI-HEPES (dialysis performed with 10 kDa MWCO membrane then filtered sterilized). Cells were incubated in 96 well plates for one hour and then cultures were washed twice with culture medium and then resuspended in culture media and incubated as for standard merozoite invasion assay.

To test the activity of inhibitory compounds on invasion (Supporting Information Table 3), E64 treated schizonts were resuspended and filtered in culture medium without human serum. A typical merozoite invasion inhibition assay consisted of filtered merozoites (40 ul) added to RBC suspension (5 ul at 12% hematocrit; final concentration 1% hematocrit) and inhibitory compounds (5 ul at 10-times the final concentration required) in 96-well plates. Merozoite:RBCs suspensions containing inhibitors were incubated for one hour and then washed twice with culture medium to remove inhibitors, then resuspended in fresh culture media and incubated and analysed as for standard merozoite invasion assays. A number of compounds were also tested for inhibition of schizont rupture by incubating late stage schizonts (1-2% parasitaemia, 1% haematocrit in 50 ul) with a 1 in 10 dilution of compounds and monitoring the course of schizont rupture at 3 time points over an 8 hour period by flow cytometry.

### Example 9

### Merozoite Purification and Invasion Inhibition Assays

Previous studies report that merozoites collected from spontaneously ruptured schizonts, usually several hours post-rupture, retain little or no invasive capacity. Applicant has explored whether mature schizonts could be ruptured, and merozoites purified. Highly synchronous mature-stage parasites were isolated (to around 95% purity), returned to culture and monitored for rupture. Once rupture began to occur, whole parasite preparations were passed through a 1.2 um filter to rupture schizonts and isolate free merozoites. Culture of the merozoite preparation with fresh RBCs confirmed that a proportion retained invasive capacity, as indicated by the presence of developing intra-erythrocytic parasites (data not shown).

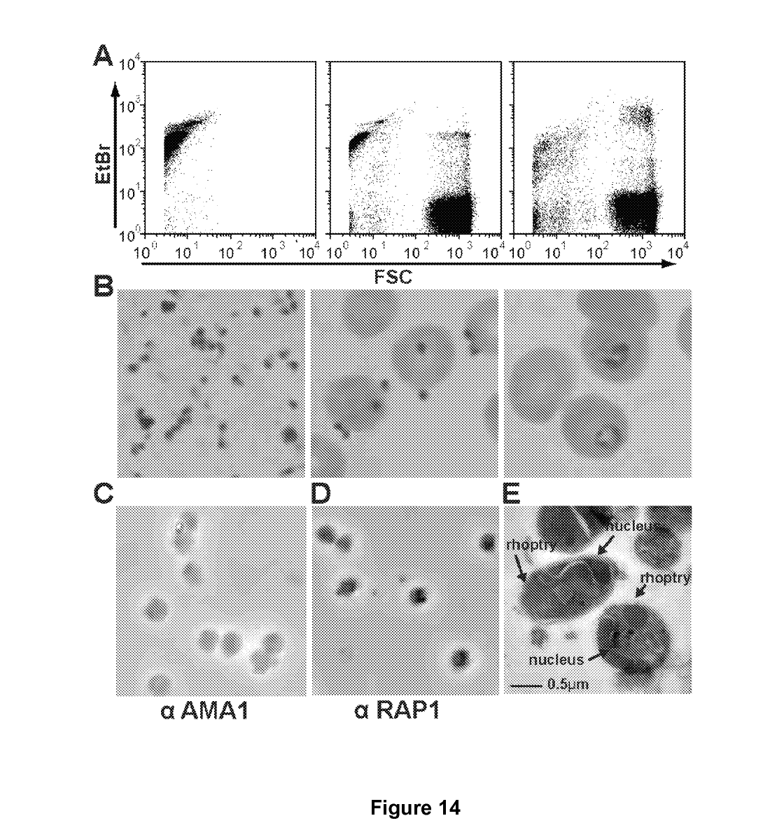

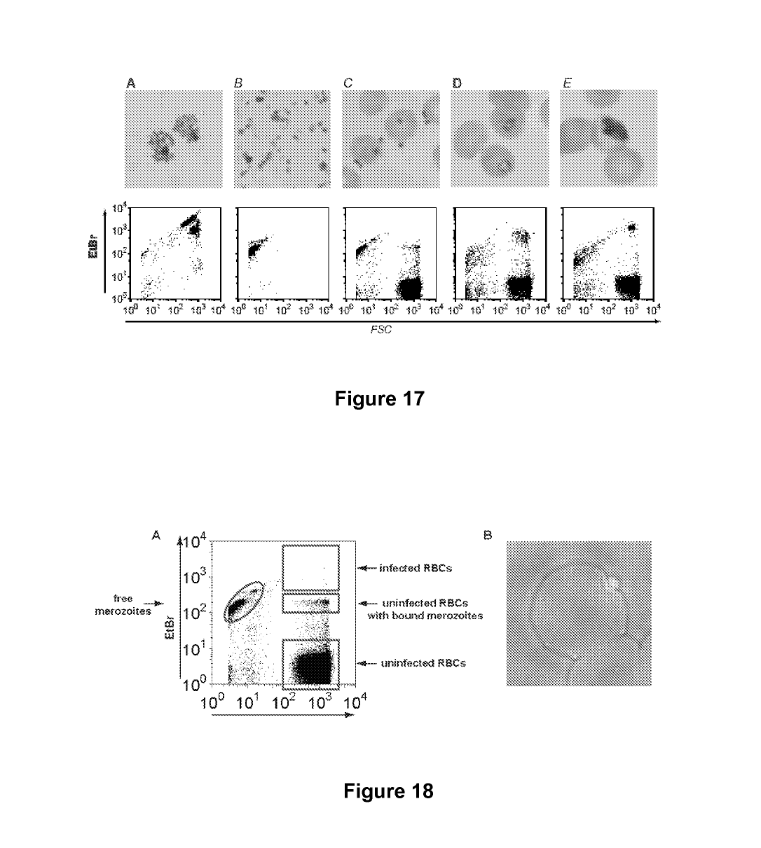

In order to increase the yield of merozoites that retained their invasive potential, purified mature-stage parasites were treated with the protease inhibitor trans-Epoxysuccinyl-L-leucylamido(4-guanidino)butane (E64), which prevents merozoite release from schizonts by inhibiting rupture (FIG. 17). Applicant found that incubating merozoites with E64 did not affect their invasive capacity. This approach enabled a parasite preparation enriched for schizonts to be obtained. After the majority of parasites were fully developed in the presence of E64, parasites were pelleted and resuspended into a small volume of culture media and merozoites were purified by filtration. Without wishing to be limited by theory, analysis of the filtrate by Giemsa-stained smears and flow cytometry (which allows for populations of E64 treated schizonts, uninfected RBCs, free merozoites, RBCs with bound merozoites and infected RBCs to be differentiated, FIG. 14A) suggests that filtration completely disrupts schizonts and excludes parasitized and non-parasitized RBCs resulting in a preparation containing only merozoites and hemozoin crystals. When added to uninfected RBCs, purified merozoites were able to bind to uninfected RBCs and a proportion of merozoites invaded (FIG. 14B,C; and FIG. 17). Analysis after 24 and 48 hours of culture showed the presence of highly synchronous parasites with normal development (FIG. 14B,C; FIG. 17). Following parasites post-invasion suggests that most or all merozoites that do successfully invade then develop normally through to mature stages. Direct comparison of merozoites purified from untreated or E64-treated schizonts showed that merozoites from E64-treated preparations had a surprisingly higher proportion of merozoites that invaded (2.1±0.65 [mean±SEM] times higher). Applicant typically found that 100 ml of parasite culture (3% hematocrit, 3% parasitemia) gave a yield of 4×10⁸ merozoites.

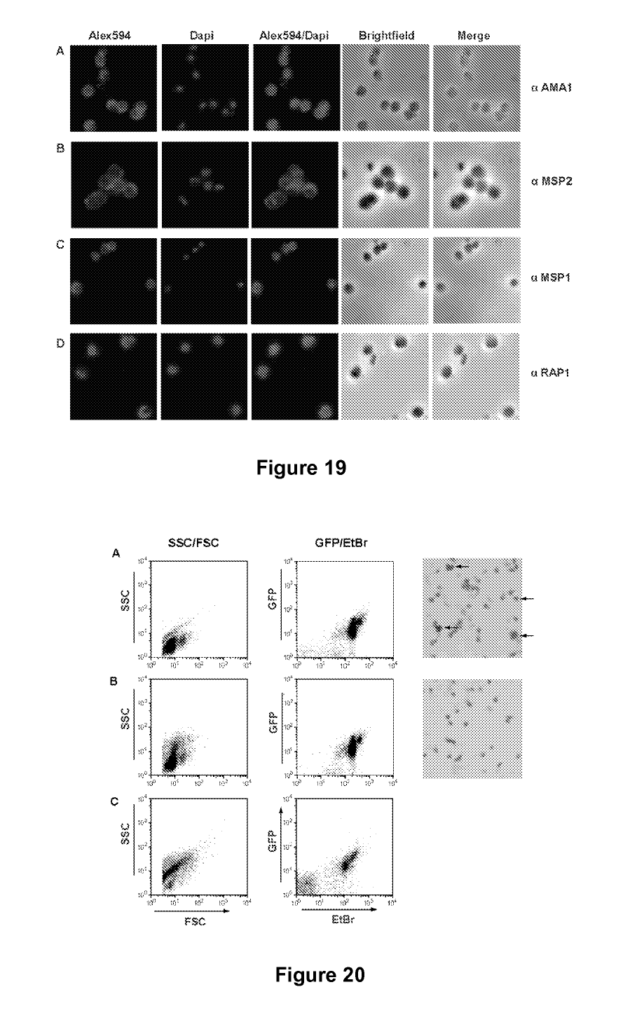

The integrity of purified merozoites was assessed by immunofluorescence microscopy (IF) and transmission electron microscopy (TEM). By IF, a high proportion of merozoites were positive with antibodies to merozoite surface proteins; >90% were positive for AMA1 and MSP1-19; 70% for MSP2. Anti-AMA1 labeled the whole merozoite surface, confirming that AMA1 is released from the micronemes and redistributes over the merozoite surface post-release (FIG. 14C; FIG. 19). Antibodies to RAP1 showed an apical staining pattern, suggesting that rhoptry proteins involved in invasion had not yet been released (FIG. 14D; FIG. 19). EM further confirmed that the majority of purified merozoites were intact and that organelles and key structures were preserved (FIG. 14E).

Experiments were performed with a GFP-expressing D10-PfPHG parasite line to facilitate identification and tracking of merozoites and invasion events by flow cytometry and microscopy. Furthermore; the methods of the present invention have been successfully used to isolate merozoites and obtain invasion of RBCs for the parental D10 and 3D7 lines. The majority of merozoite invasion events resulted in singly-infected RBCs (2% of infected RBCs were multiply-infected compared with 26% in standard culture conditions using equivalent parasitemias and hematocrits [3 assays in duplicate]).

Applicant has also demonstrated hemozoin crystals can be removed from merozoite preparations by passage over a magnet column (FIG. 20). The removal of haemozoin is considered important for certain applications, such as use in assays of cellular immune responses.

Applicant has also demonstrated effective pelleting of merozoites by centrifugation. The majority of merozoites were pelleted at 2000×g. Viability of pelleted merozoites was maintained (50% of non-centrifuged controls, data not shown).

Applicant has demonstrated herein that the efficiency of merozoite invasion was significantly influenced by the ratio of merozoites to cells. As the merozoite:RBC ratio decreased the resulting invasion rate (proportion of merozoites that invaded) increased (FIG. 15A; FIG. 18). Maximum invasion rates were achieved at low merozoite:RBC ratios (i.e. an excess of RBCs). Under conditions of low merozoite:RBC ratios, the parasitemia of post-invasion cultures was low (FIG. 18). Higher parasitemias were achieved with high merozoite:RBC ratios (i.e. an excess of merozoites) with a subsequent decline in the proportion of merozoites that invaded.

Applicant has also demonstrated the effect of hematocrit on invasion rate. Keeping the merozoite:RBC ratio fixed, the invasion rate increased in relation to increasing hematocrit (FIG. 18) (e.g. invasion rate was 7.1, 7.7, 8.8% at RBC concentrations of 113, 226, 340×10³/ul, respectively; one representative experiment). Without wishing to be limited by theory, the demonstration that increasing the relative concentration of merozoites resulted, in a lower proportion of merozoites invading suggests that there may be a period of competitive exclusion or interference by merozoites that is limiting invasion of merozoites. Alternatively, the number of RBCs that support efficient invasion may be limited; however, high parasitemias are achievable in standard culture suggesting that RBC receptiveness may not be the major factor.

Because low merozoite:RBC ratios resulted in low parasitemias in the first growth cycle, Applicant has demonstrated that the requirements for invasion efficiency and resulting parasitemia can be controlled. Accordingly, the methods of the present invention may be performed at 2% final hematocrit and high merozoite:RBC ratios, to balance these factors and to obtain high parasitemias for analysis (e.g. FACS analysis), inhibition studies, and imaging.

Applicant has demonstrated that using the methods of the present invention, it is possible to obtain consistently surprisingly high efficiency of merozoite invasion. For example, the proportion of merozoites invading was 17.7%, 16.5% and 14.9% in three experiments. For comparison, the invasion rate of D10-PfPHG merozoites in standard in vitro cultures was estimated at 20-40% of merozoites invading RBCs based on the assumption of 16 merozoites per schizont and an observed asexual replication rate of D10-PfPHG of 4 to 7-fold per cycle.

It may be possible to obtain higher invasion rates using purified merozoites if the synchronicity of parasite populations prior to filtration can be further improved. Applicant has demonstrated such methods as described herein. For example, heparin or a sulfated polysaccharide molecule capable of inhibiting invasion may be added at a predetermined time post invasion to stop the invasion process, thereby resulting in a population of parasites synchronqus within the period of time for which invasion was allowed to occur.

Prior art methods involve examining invasion under static conditions. Applicant has determined that agitation of merozoite:RBC suspensions, to promote mixing, increased the invasion rate. Agitation of cell suspensions (400 rpm on a plate-shaker) for 10 mins after mixing merozoites and RBCs increased the invasion rate and resulting parasitemia by 4.9 +/−1.3 fold (mean±SEM; 7 assays in triplicate). Of the merozoites that did not invade, a proportion was visibly bound to the RBC surface and the remainder persisted as free merozoites (FIG. 14B). Similarly, free merozoites and RBC-bound merozoites could also be observed in standard in vitro culture. It was possible to isolate some RBCs with bound merozoites by flow cytometry and cell sorting (FIG. 15). However, a high proportion of RBCs sorted had no evidence of bound merozoites, possibly because binding of merozoites to RBCs was weak or transient and interactions were not sustained during cell sorting despite efforts to fix the samples.

In order to determine the kinetics of survival, merozoites were incubated in culture medium on ice, at 22° C., or at 37° C. for different times after purification from schizonts. Merozoites were then mixed with RBCs and incubated at 37° C. to allow invasion to occur. After pre-incubation at 37° C., the invasive potential of merozoites decreased rapidly, with a half-life of approximately 5 minutes (FIG. 15B), and was similar after incubation on ice. At room temperature, the invasive half-life was increased to approximately 15 minutes; this longer survival has important practical value for studies of merozoite biology and inhibitors, allowing sufficient time to perform treatments or manipulations of merozoites prior to testing their invasive capacity. At 40° C., invasion was greatly reduced to only 16.5 +1-3% (mean±SEM; 3 experiments in duplicate) of the level of invasion observed at 37° C., suggesting that high fevers associated with malaria may impact on parasite replication in the blood stream.

To define the kinetics of merozoite invasion, Applicant used the invasion-inhibitor heparin to block invasion so that invasion was allowed to occur for defined periods of time. Merozoites and RBCs were co-incubated and heparin was subsequently added at different time points to stop any further invasion. We found that invasion occurred at a steadily increasing rate and >80% of maximal invasion by merozoites occurred in the first 10 minutes (FIG. 15C). Although agitation of merozoite:RBC suspensions increased the proportion of merozoites that invaded, (described above), it had little effect on the rate of invasion over time; the proportion of total invasion that occurred in the first 5 min was the same as static conditions, and was only 16% greater at 10 minutes. Since the majority of purified merozoites invade within the first 10 minutes of mixing with RBCs it is possible with this method to obtain cultures that have a much tighter synchronicity than achieved with commonly used methods of synchronization; the addition of heparin can be used to exclude any potential later invasion events without adversely affecting intra-erythrocytic development.

Prior to the present invention, it was not known whether invasion requires, or is enhanced by serum components. E64-treated schizonts were washed and filtered in protein free RPMI-HEPES and then added to RPMI-HEPES with different concentrations of human serum (heat inactivated, pooled from non-exposed donors); serum was used with or without dialysis against RPMI-HEPES (10,000 MWCO membrane). Merozoites were allowed to invade RBCS for 1.hour, and cells were then washed and returned to normal culture conditions (FIG. 15D). Interestingly, merozoite invasion was maximal in serum-free RPMI-HEPES, indicating that invasion does not require serum and is not enhanced by serum. Invasion rates at concentrations of up to 10% dialyzed serum or non-dialyzed serum were similar to the invasion rate in serum-free conditions; 10% is a serum concentration typically used for standard in vitro culture. The invasion rate was substantially lower in the presence of high concentrations of non-dialyzed serum (69% reduction with serum at 80%). However, invasion into dialyzed serum occurred efficiently even at high concentrations (in 80% serum invasion was 80% compared to no serum). This means that studies of invasion efficiency or inhibition assays can be performed using antibodies or serum components at concentrations that are close to those in vivo, but can also be performed under conditions that require an absence of serum components or protein.

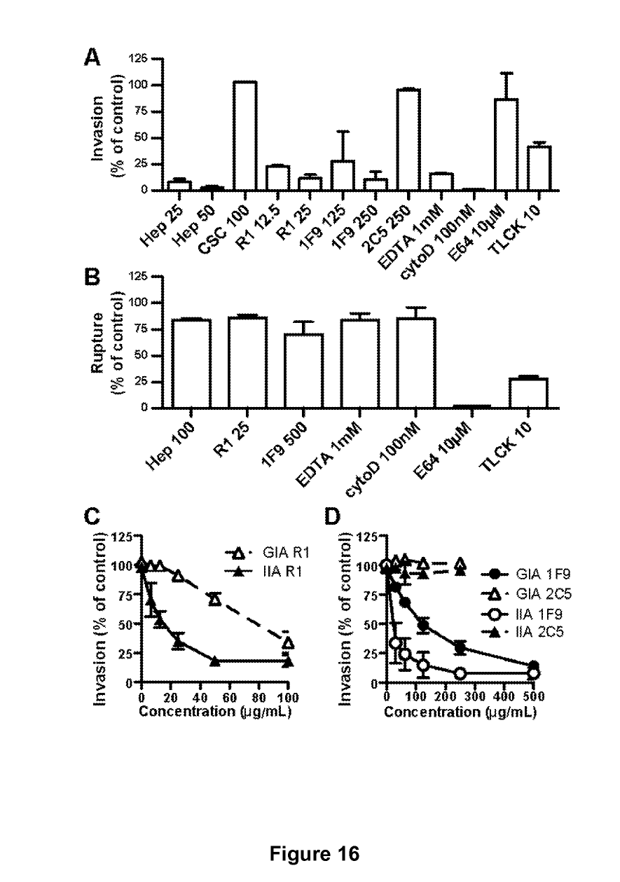

Presently, there are no assays to specifically measure inhibition of invasion. Therefore, Applicant developed an invasion-inhibition assay (IIA) based on these methods that would be suitable for high throughput testing of antibodies and novel compounds using small volume microtitre plates and evaluating invasion using flow cytometry. Using this assay, known invasion inhibitory compounds (Table 15), heparin, AMA1-binding peptide R1, and cytochalasin D effectively inhibited merozoite invasion (FIG. 16A). EDTA also inhibited invasion, presumably by interfering with calcium flux, which is thought to be essential for invasion. The inhibitory anti-AMA1 MAb 1F9 also effectively inhibited invasion, whereas MAb 2C5 did not.

Future patent applications may be filed on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions. Finally, it is to be understood that various other modifications and/or alterations may be made without departing from the spirit of the present invention as outlined herein.

### REFERENCES CITED

1. A method for identifying antiplasmodial activity in a candidate sulfated polysaccharide molecule, the polysaccharide molecule comprising two or more disaccharide units, the method comprising one or more of the following steps:
(i) assessing the average degree of sulfation per disaccharide unit,
(ii) assessing the position of two or more sulfate groups in at least one of the disaccharide units,
(iii) assessing the linkage of one or more sulfate groups in at least one of the disaccharide units,
(iv) assessing the saccharide backbone composition,
wherein the candidate molecule is considered to possess antiplasmodial activity if one or more of the following conditions is satisfied:
(a) the average degree of sulfation is at least about 1 sulfate group per disaccharide unit
(b) 2 or more sulfate groups are present on a single monosaccharide residue of the disaccharide unit
(c) the 50% or more of sulfate groups are O-linked
(d) the saccharide backbone comprises 50% or less iduronic acid.

2. The method according to claim 1 comprising any two or more of steps (i), (ii), (iii) or (iv).

3. The method according to claim 1 comprising steps any three or more of steps (i), (ii), (iii) or (iv).

4. The method according to claim 1 comprising steps (i), (ii), (iii) and (iv).

5. (canceled)

6. The method according to claim 1 wherein the average degree of sulfation is at least about 2 sulfate groups per disaccharide unit.

7. (canceled)

8. (canceled)

9. (canceled)

10. (canceled)

11. (canceled)

12. (canceled)

13. The method according to claim 1 wherein the polysaccharide molecule is considered to have antiplasmodial activity if the 50% or more of sulfate groups is O-linked.

14. The method according to claim 1 comprising the step of assessing the saccharide backbone composition.

15. The method according to claim 14 wherein the polysaccharide molecule is considered to have antiplasmodial activity if the saccharide backbone composition comprises 50% or less of iduronic acid.

16. The method according to claim 1 wherein the sulfated polysaccharide consists of at least 5 disaccharide units.

17. (canceled)

18. (canceled)

19. (canceled)

20. A sulfated polysaccharide molecule identified by the method according to claim 1.

21. A method for producing or rationally designing a sulfated polysaccharide having antiplasmodial activity, the method comprising the steps of providing a polysaccharide molecule or a sulfated polysaccharide molecule having two or more disaccharide units, modifying the polysaccharide or sulfated polysaccharide molecule by one or more of the following steps:
(i) alter or ensure the average degree of sulfation is at least about 1 sulfate group per disaccharide unit,
(ii) alter or ensure the position of sulfation is such that 2 or more sulfate groups are present on a single monosaccharide residue of a disaccharide unit,
(iii) alter or ensure the linkage of sulfation is such that 50% or more of sulfate groups are O-linked,
(iv) alter or ensure that the saccharide backbone composition comprises 50% or less iduronic acid.

22. The method according to claim 20 comprising any two or more of steps (i), (ii), (iii) or (iv).

23. The method according to claim 20 comprising any three or more of steps (i), (ii), (iii) or (iv).

24. The method according to claim 20 comprising steps (i), (ii), and (iii), and (iv).

25. (canceled)

26. The method according to claim 21 wherein the average degree of sulfation is at least about 2 sulfate groups per disaccharide unit.

27. (canceled)

28. (canceled)

29. (canceled)

30. (canceled)

31. (canceled)

32. (canceled)

33. The method according to claim 21 wherein the polysaccharide molecule is considered to have antiplasmodial activity if 50% or more of sulfate groups is O-linked.

34. The method according to claim 21 comprising the step of assessing the saccharide backbone composition.

35. The method according to claim 34 wherein the polysaccharide molecule is considered to have antiplasmodial activity if the saccharide backbone composition comprises 50% or less iduronic acid.

36. The method according to claim 21 wherein the sulfated polysaccharide consists of at least 5 disaccharide units.

37. (canceled)

38. (canceled)

39. (canceled)

40. A sulfated polysaccharide molecule produced or rationally designed according to the method of claim 21.

41. A composition comprising a sulfated polysaccharide molecule according to claim 40 and a pharmaceutically acceptable carrier.

42. A method for treating or preventing an infection with a Plasmodium, the method comprising administering to a subject in need thereof an effective amount of a composition according to claim 41, wherein the sulfated polysaccharide molecule is not a compound selected from the group consisting of heparin, heparin sulfate, pentosan polysulfate, dextran sulfate, curdlan sulfate, cellulose sulfate, a carrageen, periodate treated heparin, and fucoidan.

43. The method according to claim 42 wherein the Plasmodium is selected from the group consisting of Plasmodium falciparum, Plasmodium malariae, Plasmodium ovale, Plasmodium vivax and Plasmodium knowlesi.

44. The method according to claim 42 wherein the Plasmodium is Plasmodium falciparum.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)
